SEQUENCE LISTING (38 sequences)

SEQ ID NO: 1 - protein, 38 aa, P. tetraurelia
FAASEMQGWRNTMEDAHIHRHDIIQDVSVFGVFDGHGG

SEQ ID NO: 2 - protein, 53 aa, A. thaliana
YGFTSICGRRPEMEDAVSTIPRFLQSSSGSMLDGRFDPQSAAHFFGVYDGHGG

SEQ ID NO: 3 - protein, 41 aa, M. mulatta
YGLSSMQGWRVEMEDAHTAVIGLPSGLESWSFFAVYDGHAG

SEQ ID NO: 4 - protein, 41 aa, H. Sapiens; identical to SEQ ID NO: 3

SEQ ID NO: 5 - protein, 41 aa, G. gallus
YGLSSMQGWRVEMEDAHTAVVGIPHGLEDWSFFAVYDGHAG

SEQ ID NO: 6 - protein, 43 aa, C. elegans
YGMSSMQGWRICMEDSHIAEAIMSQSSPYKDWSFFAVFDGHAG

SEQ ID NO: 7 - protein, 46 aa, D. rerio
TSIHAIKNMRRKMEDKHVVIPDFNTLFNLQDQEEQAYFAVFDGHGG

SEQ ID NO: 8 - protein, 46 aa, M. musculus
VSIHAIRNTRRKMEDRHVSLPAFNHLFGLSDSVHRAYFAVFDGHGG

SEQ ID NO: 9 - protein, 48 aa, R. norvegicus
GYVAERKGEREEMQDAHVILNDITQECNPPSSLITRVSYFAVFDGHGG

SEQ ID NO: 10 - protein, 40 aa, S. pombe
LMEDKNQRWRRSMEDTHICLYDFGGNQDDGFVAVYDGHAG

SEQ ID NO: 11 - protein, 221 aa, A. thaliana
MANSESSSSPVNEEENSQRISTLHHQTMPSDLTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNRNNNNNNSSQVR

SEQ ID NO: 12 - protein, 434 aa, A. thaliana
MEEVSPAIAGPFRPFSETQMDFTGIRLGKGYCNNQYSNQDSENGDLMVSLPETSSCSVSGSHGSESRKVLISRINSPNLNMKESAAADIVVVDISAGDEINGSDITSEKKMISRTESRSLFEFKSVPLYGFTSICGRRPEMEDAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLKPRRKLKSKPLN

SEQ ID NO: 13 - DNA, 35 bp, Artificial Sequence
  synthetic oligonucleotide
ccgacaacgcgtgtgcctttgtatggttttacttc

SEQ ID NO: 14 - DNA, 64 bp, Artificial Sequence
  synthetic oligonucleotide
ccgacagcggccgctcacttatcgtcatcgtccttgtaatccttcaaatcaaccaccaccacac

SEQ ID NO: 15 - DNA, 39 bp, Artificial Sequence
  synthetic oligonucleotide
ccgacagtcgacgccaccatggtgagcaagggcgaggag

SEQ ID NO: 16 - DNA, 35 bp, Artificial Sequence
  synthetic oligonucleotide
ccgacaggcgcgcccttgtacagctcgtccatgcc

SEQ ID NO: 17 - DNA, 37 bp, Artificial Sequence
  synthetic oligonucleotide
ccgacaggcgcgccaactcaagacgaattcacccaac

SEQ ID NO: 18 - DNA, 67 bp, Artificial Sequence
  synthetic oligonucleotide
ccgacagcggccgctcaagcgtaatctggaacatcgtatgggtagttcatagcttcagtgatcgaag

SEQ ID NO: 19 - DNA, 36 bp, Artificial Sequence
  synthetic oligonucleotide
ccgacagtcgacatgaacaaactacggcaaagcttc

SEQ ID NO: 20 - DNA, 58 bp, Artificial Sequence
  synthetic oligonucleotide
ccgacaacgcgtacccccaccagaacccccaccagaaagttctatttcaaacgttttc

SEQ ID NO: 21 - DNA, 34 bp, Artificial Sequence
  synthetic oligonucleotide
ccgacaacgcgtaatggggctacatgtcttctga

SEQ ID NO: 22 - DNA, 36 bp, Artificial Sequence
  synthetic oligonucleotide
ccgacagcggccgcaatggggctacatgtcttctga

SEQ ID NO: 23 - DNA, 42 bp, Artificial Sequence
  synthetic oligonucleotide
ccgacagtcgacgccaccatgcaggcgcagcagctgccctac

SEQ ID NO: 24 - DNA, 36 bp, Artificial Sequence
  synthetic oligonucleotide
ccgacaggcgcgccggaagaatgggcattctccaac

SEQ ID NO: 25 - DNA, 86 bp, Artificial Sequence
  synthetic oligonucleotide
ccgacagcggccgctcatacctttctcttcttttttggatctacctttctcttcttttttggatcgttcaagggtttgctcttgag

SEQ ID NO: 26 - DNA, 68 bp, Artificial Sequence
  synthetic oligonucleotide
ccgacagaattcgccaccatgggtagcaacaagagcaagggaggtgtgcctttgtatggttttacttc

SEQ ID NO: 27 - DNA, 39 bp, Artificial Sequence
  synthetic oligonucleotide
ccgacaggcgcgcctggaatgtggcatgaaggcctggaa

SEQ ID NO: 28 - DNA, 41 bp, Artificial Sequence
  synthetic oligonucleotide
ccgacagcggccgctcactgctttgagattcgtcggaacac

SEQ ID NO: 29 - DNA, 43 bp, Artificial Sequence
  synthetic oligonucleotide
ccgacagaattcgccaccatgggccctaaaaagaagcgtaaag

SEQ ID NO: 30 - DNA, 39 bp, Artificial Sequence
  synthetic oligonucleotide
ccgacaggcgcgcctcccaccgtactcgtcaattccaag

SEQ ID NO: 31 - DNA, 34 bp, Artificial Sequence
  synthetic oligonucleotide
ccgacagagctcatgaagctactgtcttctatcg

SEQ ID NO: 32 - DNA, 35 bp, Artificial Sequence
  synthetic oligonucleotide
ccgacaacgcgtcgatacagtcaactgtctttgac

SEQ ID NO: 33 - DNA, 85 bp, Artificial Sequence
  synthetic oligonucleotide
ccgacaacgcgtatggtgcctttgtatggttttacttcgatttgtggaagaagacctgagatggaagctgctgtttcgactatac

SEQ ID NO: 34 - DNA, 35 bp, Artificial Sequence
  synthetic oligonucleotide
ccgacaggatccgtgcctttgtatggttttacttc

SEQ ID NO: 35 - DNA, 38 bp, Artificial Sequence
  synthetic oligonucleotide
ccgacaggatcctcacttcaaatcaaccaccaccacac

SEQ ID NO: 36 - DNA, 34 bp, Artificial Sequence
  synthetic oligonucleotide
ccgacaactagtctgtggaatgtgtgtcagttag

SEQ ID NO: 37 - DNA, 34 bp, Artificial Sequence
  synthetic oligonucleotide
ccgacagaattccgaaaatggatatacaagctcc

SEQ ID NO: 38 - DNA, 38 bp, Artificial Sequence
  synthetic oligonucleotide
ccgacaggatcctcaagcgtaatctggaacatcgtatg

(19) United States
(12) Patent Application Publication
(10) Pub. No.: US 2013/0158098 A1
(43) Pub. Date: Jun. 20, 2013

(54) Alkenyl Substituted Cycloaliphatic Compounds as Chemical Inducers of Proximity

(71) Applicants: Fu-Sen Liang, Mountain View, CA (US); Gerald R. Crabtree, Woodside, CA (US)

(72) Inventors: Fu-Sen Liang, Mountain View, CA (US); Gerald R. Crabtree, Woodside, CA (US)

(73) Assignee: The Board of Trustees of the Leland Standford Junior University, Palo alto, CA (US)

(21) Appl. No.: 13/704,517

(22) PCT Filed: Jun. 15, 2011
(86) PCT No.: PCT/US11/40503

§ 371 Date: Mar. 5, 2013

Related U.S. Application Data
(60) Provisional application No. 61/357,035, filed on Jun. 21, 2010.

(51) Int. Cl. A61K 31/19; C12N 5/071

(52) U.S. Cl. CPC A61K 31/19; C12N 5/0602
USPC 514/44 A; 435/375; 536/23.1; 514/557

(57) ABSTRACT

Methods of inducing proximity of chimeric molecules in a cell are provided. Aspects of the methods include contacting a cell with an amount of alkenyl substituted cycloaliphatic (ASC) inducer compound, e.g., abscisic acid, effective to induce proximity of first and second chimeric molecules. Also provided are compositions and kits for practicing various embodiments of the methods. Methods of the invention find use in a variety of different applications, including transcription induction applications.

### CROSS-REFERENCE TO RELATED APPLICATIONS

Pursuant to 35 U.S.C. §119 (e), this application claims priority to the filing date of U.S. Provisional Patent Application Ser. No. 61/357,035 filed Jun. 21, 2011; the disclosure of which is herein incorporated by reference.

### ACKNOWLEDGMENT OF GOVERNMENT SUPPORT

This invention was made with Government support under Grant Nos. NS046789, AI060027, HD055391 from the National Institutes of Health. The Government has certain rights in this invention.

### INTRODUCTION

The development of small molecules that modulate protein function in a tunable fashion has been a major focus in chemical biology. Chemical inducers of proximity are cell-permeable molecules capable of inducing proximity of two distinct biological entities, e.g., proteins, and were first described in Spencer D. M.; Wandless T. J.; Schreiber S. L.; Crabtree G. R. Science 1993, 262, 1019. The importance of proximity in regulation of biologic processes is illustrated by the domainal nature of protein structure and the frequency with which these domains recruit the protein (i.e. induce proximity) to nucleic acids, lipid membranes or other proteins. Small molecule mediated mechanisms to control proximity may be employed to control a wide range of biologic processes. Induced proximity has been a highly useful way of controlling many biologic processes including signaling, transcription, protein degradation, membrane and nuclear localization as well as many others.

The usefulness of small molecule induced proximity has been limited by the fact that all of the small molecules known to the inventors that have been used to date to induce proximity are at least one of toxic, unstable or difficult to synthesize in large enough quantities for routine use. For example, rapamycin and FK506 are both excellent inducers of dimerization or proximity, but produce too many side effects through their interactions with calcineurin or mTOR to be useful for many in vitro investigative needs and are too toxic for gene therapeutic applications in humans or animals. Modifications of rapamycin, such as C20 methyl rapamycin, are not toxic but are so unstable as to be generally not useful (Stankunas K., Bayle J. H., Gestwicki J. E., Lin Y. M., Wandless T. J., and Crabtree G. R., Mol Cell 2003, 12, 1615-1624; Bayle J. H., Grimley J. S., Stankunas K., Gestwicki J. E., Wandless T. J., and Crabtree G. R., Chem. Biol. 2006, 13, 99-107). Other modifications, such as AP1510 or AP1903 (Amara J. F., Clackson T., Rivera V. M., Guo T., Keenan T., Natesan S., Pollock R., Yang W., Courage N. L., Holt D. A., and Gilman M., Proc Natl Acad Sci USA 1997, 94, 10618-10623; Clackson T., Yang W., Rozamus L. W., Hatada M., Amara J. F., Rollins C. T., Stevenson L. F., Magari S. R., Wood S. A., Courage N. L., Lu X., Cerasoli F., Jr., Gilman M., and Holt D. A., Proc Natl Acad Sci USA 1998, 95, 10437-10442), are too expensive for routine use. In addition, each of these molecules is limited in its application by high-affinity interactions with endogenous molecules that dilute out the effective dimer pairs induced in a cell.

### SUMMARY

Methods of inducing proximity of chimeric molecules in a cell are provided. Aspects of the methods include contacting a cell with an amount of an alkenyl substituted cycloaliphatic (ASC) inducer compound, e.g., abscisic acid, effective to induce proximity of at least first and second chimeric molecules. Also provided are compositions and kits for practicing various embodiments of the methods. Methods of the invention find use in a variety of different applications, including transcription induction applications.

Aspects of the invention include methods of inducing proximity of first and second chimeric molecules in a cell. In certain embodiments, the methods include: contacting the cell with an amount of an alkenyl substituted cycloaliphatic (ASC) inducer compound effective to induce proximity of the first and second chimeric molecules, wherein the first and second chimeric molecules each comprise an ASC inducer domain and an effector domain. In some instances, the ASC inducer compound is non-toxic and/or may have a molecular weight of 500 daltons or less. In some instances, the ASC inducer compound comprises a cycloaliphatic ring substituted with a hydroxyl and/or oxo group, for example an ASC inducer compound is described by the formula:

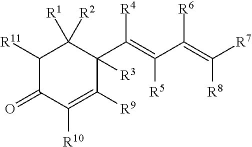

wherein R¹, R², R³, R⁴, R⁵, R⁶, R⁷, R⁸, R⁹, R¹⁰ and R¹¹ are independently selected from hydrogen, an alkyl, an aryl, an alkenyl, an alkynyl, a carbonyl, an acyl, a halogen, a hydroxy, an alkoxy, an aryloxy, and a heterocyclic group and any two of R¹, R², R³, R⁴, R⁵, R⁶, R⁷, R⁸, R⁹, R¹⁰ and R¹¹ can optionally be cyclically linked, e.g., abscisic acid. In some embodiments, the first and second chimeric molecules are chimeric proteins. In some instances, the ASC inducer domain of the first chimeric protein is an ASC inducer compound specific binding domain, e.g., a PYR abscisic acid binding domain, for example, a PP2C domain, such as a PP2C domain lacks phosphatase activity. In some instances, the effector domains of the first and second chimeric molecules are different. In some instances, the ASC inducer mediated association of the effector domains causes a cellular activity, e.g., a signal, such as a transcription signal. In some instances, the first chimeric molecule is a DNA binding domain and the effector domain of the second chimeric molecule is a transcription activation domain. In some instances, the effector domain of the first chimeric molecule is a cellular localization domain and the effector domain of the second chimeric molecule is a member of a signaling pathway. In some instances, the cell is in vitro. In some instances, the method includes administering the cell to a multi-cellular organism following contact of the cell with the ASC inducer compound. In some instances, the cell is a part of a multi-cellular organism and the method comprises administering the ASC inducer compound specific binding domain to the multi-cellular organism.

Aspects of the invention also include methods of inducing transcription of a coding sequence in a cell. In some embodiments, the methods include: contacting the cell with an amount of an ASC inducer compound effective to induce proximity of first and second chimeric proteins in the cell, wherein: the first chimeric protein comprises an ASC inducer domain that specifically binds to the ASC inducer compound and a DNA binding domain; the second chimeric protein comprises an ASC inducer domain that specifically binds to the ASC inducer domain of the first chimeric protein in the presence of the ASC inducer compound and a transcription activation domain; and the ASC inducer compound mediated proximity induction of the first and second chimeric proteins results in transcription of a coding sequence that is mediated by the transcription activation domain. As described above, in some instances the ASC inducer compound is non-toxic and/or may have a molecular weight of 500 daltons or less. In some instances, the ASC inducer compound comprises a cycloaliphatic ring substituted with a hydroxyl and/or oxo group, for example an ASC inducer compound is described by the formula:

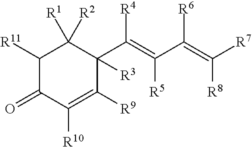

wherein R¹, R², R³, R⁴, R⁵, R⁶, R⁷, R⁸, R⁹, R¹⁰ and R¹¹ are independently selected from hydrogen, an alkyl, an aryl, an alkenyl, an alkynyl, a carbonyl, an acyl, a halogen, a hydroxy, an alkoxy, an aryloxy, and a heterocyclic group and any two of R¹, R², R³, R⁴, R⁵, R⁶, R⁷, R⁸, R⁹, R¹⁰ and R¹¹ can optionally be cyclically linked, e.g., abscisic acid. As described above, in some instances, the ASC inducer domain of the first chimeric protein is an ASC inducer compound specific binding domain, e.g., a PYR abscisic acid binding domain, for example, a PP2C domain, such as a PP2C domain lacks phosphatase activity. In some instances, the cell is in vitro. In some instances, the method includes administering the cell to a multi-cellular organism following contact of the cell with the ASC inducer compound. In some instances, the cell is a part of a multi-cellular organism and the method comprises administering the ASC inducer compound specific binding domain to the multi-cellular organism.

Aspects of the invention further include nucleic acids that include a coding sequence for a chimeric protein that comprises ASC inducer domain and an effector domain. In some instances, the ASC inducer domain of the first chimeric protein is an ASC inducer compound specific binding domain, e.g., a PYR abscisic acid binding domain, for example, a PP2C domain, such as a PP2C domain lacks phosphatase activity. Effector domains may vary, where effector domains of interest include DNA binding domains, transcription activation domains, etc. In some instances, the nucleic acid comprises a coding sequence for two chimeric proteins each comprising an ASC inducer domain and an effector domain. In some instances, the nucleic acid is present in a vector. In some instances, the nucleic acid is present in a cell, where in certain of these embodiments the cell is part of a non-human multi-cellular organism.

Aspects of the invention further include kits that include: first and second nucleic acids each encoding a chimeric protein that comprises an ASC inducer domain and an effector domain, e.g., as described above, and an ASC inducer compound, e.g., as described above.

Aspects of the invention further include a composition comprising: an ASC inducer compound; and a pharmaceutically acceptable vehicle. As described above, in some instances the ASC inducer compound is non-toxic and/or may have a molecular weight of 500 daltons or less. In some instances, the ASC inducer compound comprises a cycloaliphatic ring substituted with a hydroxyl and/or oxo group, for example an ASC inducer compound is described by the formula:

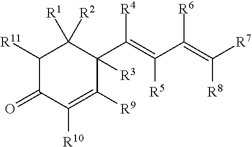

wherein R¹, R², R³, R⁴, R⁵, R⁶, R⁷, R⁸, R⁹, R¹⁰ and R¹¹ are independently selected from hydrogen, an alkyl, an aryl, an alkenyl, an alkynyl, a carbonyl, an acyl, a halogen, a hydroxy, an alkoxy, an aryloxy, and a heterocyclic group and any two of R¹, R², R³, R⁴, R⁵, R⁶, R⁷, R⁸, R⁹, R¹⁰ and R¹¹ can optionally be cyclically linked, e.g., abscisic acid.

Aspects of the invention further include methods of determining whether an ASC inducer compound, e.g., as described above, is present in a sample. In some instances, the method includes: (a) contacting the sample with cell that: (i) expresses first and second chimeric proteins each comprising an ASC inducer domain and an effector domain (e.g., as described above); and (ii) comprises a reporter construct whose transcription is activated upon proximity induction of the effector domains of the chimeric molecules; and (b) evaluating expression of the reporter construct to determine whether the ASC inducer compound is present in the sample. In some instances, the sample is a serum sample, e.g., one that is obtained from a laboratory animal or a human patient.

Aspects of the invention further include kits that include: (a) a cell that: (i) expresses first and second chimeric proteins each comprising an ASC inducer domain and an effector domain (e.g., as described above); and (ii) comprises a reporter construct whose transcription is activated upon proximity induction of the effector domains of the chimeric molecules; and (b) an ASC inducer compound (e.g., as described above).

### DEFINITIONS

When describing the compounds, pharmaceutical compositions containing such compounds and methods of using such compounds and compositions, the following terms have the following meanings unless otherwise indicated. It should also be understood that any of the moieties defined forth below may be substituted with a variety of substituents, and that the respective definitions are intended to include such substituted moieties within their scope.

“Acyl” refers to a —C(O)R group, where R is hydrogen, alkyl, alkenyl, cycloalkyl, heterocycloalkyl, aryl, arylalkyl, heteroalkyl, heteroalkenyl, or heteroaryl as defined herein. Representative examples include, but are not limited to, formyl, acetyl, cyclohexylcarbonyl, cyclohexylmethylcarbonyl, benzoyl, benzylcarbonyl and the like.

“Acylamino” refers to a —NR′C(O)R group, where R′ is hydrogen, alkyl, cycloalkyl, heterocycloalkyl, aryl, arylalkyl, heteroalkyl, heteroaryl, heteroarylalkyl and R is hydrogen, alkyl, alkoxy, cycloalkyl, heterocycloalkyl, aryl, arylalkyl, heteroalkyl, heteroaryl or heteroarylalkyl, as defined herein. Representative examples include, but are not limited to, formylamino, acetylamino, cyclohexylcarbonylamino, cyclohexylmethyl-carbonylamino, benzoylamino, benzylcarbonylamino and the like.

“Acyloxy” refers to the group —OC(O)H, —OC(O)-alkyl, —OC(O)-aryl or —OC(O)-cycloalkyl.

“Aliphatic” refers to hydrocarbyl organic compounds or groups characterized by a straight, branched or cyclic arrangement of the constituent carbon atoms and an absence of aromatic unsaturation. Aliphatics include, without limitation, alkyl, alkylene, alkenyl, alkynyl and alkynylene. Lower aliphatic groups typically have from 1 or 2 to 6 or 12 carbon atoms.

“Alkenyl” refers to monovalent olefinically unsaturated hydrocarbyl groups having up to about 11 carbon atoms, such as from 2 to 8 carbon atoms, and including from 2 to 6 carbon atoms, which can be straight-chained or branched and having at least 1 and including from 1 to 2 sites of olefinic unsaturation. Particular alkenyl groups include ethenyl (—CH═CH₂), n-propenyl (—CH₂CH═CH₂), isopropenyl (—C(CH₃)═CH₂), vinyl and substituted vinyl, and the like.

“Alkoxy” refers to the group —O-alkyl. Particular alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“Alkoxycarbonyl” refers to a radical —C(O)-alkoxy where alkoxy is as defined herein.

“Alkoxycarbonylamino” refers to the group —NRC(O)OR′ where R is hydrogen, alkyl, aryl or cycloalkyl, and R′ is alkyl or cycloalkyl.

“Alkyl” refers to monovalent saturated aliphatic hydrocarbyl groups particularly having up to about 12 or 18 carbon atoms, more particularly as a lower alkyl, from 1 to 8 carbon atoms and still more particularly, from 1 to 6 carbon atoms. The hydrocarbon chain may be either straight-chained or branched. This term is exemplified by groups such as methyl, ethyl, n-propyl, isopropyl, n-butyl, iso-butyl, tert-butyl, n-hexyl, n-octyl, tert-octyl and the like. The term “alkyl” also includes “cycloalkyls” as defined herein.

“Alkylene” refers to divalent saturated aliphatic hydrocarbyl groups particularly having up to about 12 or 18 carbon atoms and more particularly 1 to 6 carbon atoms which can be straight-chained or branched. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkynyl” refers to acetylenically unsaturated hydrocarbyl groups particularly having up to about 12 or 18 carbon atoms and more particularly 2 to 6 carbon atoms which can be straight-chained or branched and having at least 1 and particularly from 1 to 2 sites of alkynyl unsaturation. Particular non-limiting examples of alkynyl groups include acetylenic, ethynyl (—C≡CH), propargyl (—CH₂C≡CH), and the like.

“Amino” refers to the radical —NH₂.

“Aminocarbonyl” refers to the group —C(O)NRR where each R is independently hydrogen, alkyl, aryl or cycloalkyl, or where the R groups are joined to form an alkylene group.

“Aminocarbonylamino” refers to the group —NRC(O)NRR where each R is independently hydrogen, alkyl, aryl or cycloalkyl, or where two R groups are joined to form an alkylene group.

“Aminocarbonyloxy” refers to the group —OC(O)NRR where each R is independently hydrogen, alkyl, aryl or cycloalkyl, or where the R groups are joined to form an alkylene group.

“Aralkyl” or “arylalkyl” refers to an alkyl group, as defined above, substituted with one or more aryl groups, as defined above.

“Aryl” refers to a monovalent aromatic hydrocarbon group derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In some cases, an aryl group includes from 6 to 14 carbon atoms.

“Aryloxy” refers to —O-aryl groups wherein “aryl” is as defined herein.

“Azido” refers to a —N₃ group.

“Carbonyl” refers to —C(O)— groups, for example, a carboxy, an amido, an ester, a ketone, or an acyl substituent.

“Carboxyl” refers to a —C(O)OH group

“Cyano” refers to a —CN group.

“Cycloalkenyl” refers to cyclic hydrocarbyl groups having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like.

“Cycloalkyl” refers to cyclic hydrocarbyl groups having from 3 to about 10 carbon atoms and having a single cyclic ring or multiple condensed rings, including fused and bridged ring systems, which optionally can be substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, and multiple ring structures such as adamantanyl, and the like.

“Cycloaliphatic” refers to cyclic hydrocarbyl groups such as cycloalkenyl and cycloalkyl groups.

“Heterocycloalkyl” refers to a stable heterocyclic non-aromatic ring and fused rings containing one or more heteroatoms independently selected from N, O and S. A fused heterocyclic ring system may include carbocyclic rings and need only include one heterocyclic ring. Examples of heterocyclic rings include, but are not limited to, piperazinyl, homopiperazinyl, piperidinyl and morpholinyl.

“Halogen” or “halo” refers to fluoro, chloro, bromo and iodo.

“Hetero” when used to describe a compound or a group present on a compound means that one or more carbon atoms in the compound or group have been replaced by, for example, a nitrogen, oxygen, or sulfur heteroatom. Hetero may be applied to any of the hydrocarbyl groups described above such as alkyl, e.g. heteroalkyl, cycloalkyl, e.g. heterocycloalkyl, aryl, e.g. heteroaryl, cycloalkenyl, e.g., heterocycloalkenyl, cycloheteroalkenyl, e.g., heterocycloheteroalkenyl and the like having from 1 to 5, and particularly from 1 to 3 heteroatoms. A heteroatom is any atom other than carbon or hydrogen and is typically, but not exclusively, nitrogen, oxygen, sulfur, phosphorus, boron, chlorine, bromine, or iodine.

“Heteroaryl” refers to a monovalent heteroaromatic group derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. The heteroaryl group can be a 5-20 membered heteroaryl, or 5-10 membered heteroaryl. Particular heteroaryl groups are those derived from thiophen, pyrrole, benzothiophene, benzofuran, indole, pyridine, quinoline, imidazole, oxazole and pyrazine.

“Heterocycle” refers to organic compounds that contain a ring structure containing atoms in addition to carbon, such as sulfur, oxygen or nitrogen, as part of the ring. They may be either simple aromatic rings or non-aromatic rings. Examples include azoles, morpholine, piperazine, pyridine, pyrimidine and dioxane. The maximum number of heteroatoms in a stable, chemically feasible heterocyclic ring, whether it is aromatic or non-aromatic, is determined by factors such as, the size of the ring, the degree of unsaturation and the valence of the heteroatoms. In general, a heterocyclic ring may have one to four heteroatoms so long as the heteroaromatic ring is chemically feasible and stable.

“Hydroxyl” refers to a —OH group.

“Stereoisomer” as it relates to a given compound refers to another compound having the same molecular formula, wherein the atoms making up the other compound differ in the way they are oriented in space, but wherein the atoms in the other compound are like the atoms in the given compound with respect to which atoms are joined to which other atoms (e.g. an enantiomer, a diastereomer, or a geometric isomer). See for example, Morrison and Boyd, Organic Chemistry, 1983, 4th ed., Allyn and Bacon, Inc., Boston, Mass., p. 123.

“Substituted” refers to a group in which one or more hydrogen atoms are each independently replaced with the same or different substituent(s). “Substituted” groups particularly refer to groups having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂. Substituents of interest may include, but are not limited to, —X, —R⁸ (with the proviso that R⁸ is not hydrogen), —O—, ═O, —OR⁸, —SR⁸, —S⁻, ═S, —NR⁸R⁹, ═NR⁸, —CX₃, —CF₃, —CN, —OCN, —SCN, —NO, —NO₂, ═N₂, —N₃, —S(O)₂O⁻, —S(O)₂OH, —S(O)₂R⁸, —OS(O₂)O⁻, —OS(O)₂R⁸, —P(O)(O—)₂, —P(O)(OR⁸)(O⁻), —OP(O)(OR⁸)(OR⁹), —C(O)R⁸, —C(S)R⁸, —C(O)OR⁸, —C(O)NR⁸R⁹, —C(O)O⁻, —C(S)OR⁸, —NR¹⁰C(O)NR⁸R⁹, vNR¹⁰C(S)NR⁸R⁹, —NR¹¹C(NR¹⁰)NR⁸R⁹ and —C(NR¹⁰)NR⁸R⁹, where each X is independently a halogen.

“Sulfonyl” refers to the group —SO₂—. Sulfonyl includes, for example, methyl-SO₂—, phenyl-SO₂—, and alkylamino-SO₂—.

“Sulfinyl” refers to the group —S(O)—.

“Thioalkoxy” refers to the group —S-alkyl.

“Thioaryloxy” refers to the group —S-aryl.

“Thioketo” refers to the group ═S.

“Thiol” refers to the group —SH.

“Thio” refers to the group —S—. Thio includes, for example, thioalkoxy, thioaryloxy, thioketo and thiol.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

A subject compound may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see, e.g., the discussion in Chapter 4 of “Advanced Organic Chemistry”, 4th edition J. March, John Wiley and Sons, New York, 1992).

The term “pharmaceutically acceptable salt” means a salt which is acceptable for administration to a patient, such as a mammal (e.g., salts having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like.

The term “salt thereof” means a compound formed when the hydrogen of an acid is replaced by a cation, such as a metal cation or an organic cation and the like. Where applicable, the salt is a pharmaceutically acceptable salt, although this is not required for salts of intermediate compounds that are not intended for administration to a patient.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereoisomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 provides maps of various vectors described in the Experimental Section, below.

FIG. 2 illustrates the regulation of transcriptional activation using induced proximity by ABA. (A) Regions of PYLcs and ABIcs used (see also FIG. 1). (B) Scheme of ABA-induced luciferase activation and the design of the constructs.

FIG. 3 illustrates the effect of ABA concentration, ABA stability, and time on ABA activation of luciferase in CHO cells, as well as the reversibility of ABA activity. (A) ABA concentration dependence of luciferase activation. Different amounts of ABA were added to a CHO cell culture for 24 hours and cells were assayed for luciferase activity. The cells were pre-transfected with the activation and reporter DNA constructs for 24 hours. Induction fold change was calculated by induced over non-induced luciferase signal. (B) Time dependence of luciferase activation by ABA. 100 μM of ABA was added to the CHO cell culture and cells were assayed for luciferase activity at indicated times after ABA addition. The cells were pre-transfected with the activation and reporter DNA constructs for 24 hours. (C) The reversibility of ABA-induced luciferase activation. After 100 μM of ABA was added to the CHO cell culture for 24 hours, cells were washed with PBS and cell culture media without ABA and kept growing in ABA-free media for indicated period before assayed for luciferase activity. The cells were pre-transfected with the activation and reporter DNA constructs for 24 hours. (D) The stability of ABA in the media over cell culture. CHO cells were pre-transfected with the activation and reporter DNA constructs for 24 hours. 100 μM ABA was then added to the culture and cells were assayed for luciferase activity after 10 hours. During the 10-hour incubation, the cell culture media were replaced with media containing freshly added ABA every 1, 2.5, 5 hour or no replacement.

FIG. 4 illustrates the stability of ABA in serum. (A) ABA is stable in human serum for at least 46 hours. ABA was incubated with pure non-heat inactivated human serum at 37° C. for the indicated time period. ABA containing serum was then added to the CHO cell culture to a final 100 μM ABA concentration for 25 hours and cells were assayed for luciferase activity. The cells were pre-transfected with the activation and reporter DNA constructs for 24 hours. (B) ABA is stable in fetal calf serum. ABA was incubated with pure heat inactivated fetal bovine serum at 37° C. for indicated time period. ABA containing serum was then added to the CHO cell culture to a final 100 μM ABA concentration for 10 hours and cells were assayed for luciferase activity. The cells were pre-transfected with the activation and reporter DNA constructs for 24 hours.

FIG. 5 illustrates ABA bioavailability. (A) Intraperitoneally-administered ABA is bio-available in mice. ABA was intraperitoneally administrated to mice and serum was collected at indicated time. ABA containing serum was then added as 10% (v/v) of culture media to the CHO cell culture for 24 hours and cells were assayed for luciferase activity. The CHO cells were pre-transfected with the activation and reporter DNA constructs for 24 hours before adding serum. (B) Orally-administered ABA is bio-available in mice. ABA was orally administrated by gavage to mice and serum was collected at the indicated times. ABA containing serum was then added as 10% (v/v) of culture media to the CHO cell culture for 24 hours and cells were assayed for luciferase activity. The CHO cells were pre-transfected with the activation and reporter DNA constructs for 24 hours before adding serum.

FIG. 6 illustrates the phosphatase activity and luciferase-inducing activity of the D143A mutant of ABI. (A) Mutant ABI (D143A) lacks phosphatase activity when compared to wild type ABI. GST-ABI wt or D143A fusion proteins were expressed in E. coli. and purified by glutathione beads. A series amount of GST-ABI fusion proteins were then used for phosphatase activity assay following the protocol provided by vendor (Promega). The phosphatase activity is correlated to fluorescence intensity. (B) Mutant ABI (D143A) was comparable to wild type ABI in ABA-induced luciferase activation assay. This indicates that the mutant retains ABI-ABA-PYL binding ability. The CHO cells were pre-transfected with the activation constructs with wild type or mutant ABI and luciferase reporter DNA constructs for 24 hours. 100 μM of ABA was then added to the CHO cell culture and cells were assayed for luciferase activity after another 24 h.

FIG. 7 illustrates that ABA induced gene activation is generally applicable to various cell lines of different origins. (a) and (b) CHO cells (Chinese hamster ovary cells); (c) and (d) HEK 293 cells (human embryonic kidney cells); (e) and (f) Cos7 cells (African green monkey kidney cells); (g) and (h) NIH3T3 cells (mouse embryonic fibroblast cells); (i) and (j) TC1 cells (mouse embryonic stem cells). The cells were pre-transfected with the activation construct and GFP reporter construct for 24 hours. 100 μM of ABA was then added to the cell culture and cells were observed under fluorescence microscope for GFP expression after another 24 h.

FIG. 8 illustrates that the ABA-induced proximity system can be used to localize cellular proteins to the nucleus, cytoplasm and cell membrane to eliminate or enhance their activity. (a) and (b) GFP-PYL+Brg (nuclear)-ABI; (c) and (d) GFP-PYL+Numb (cytoplasmic)-ABI; (e) and (f) GFP-PYL+CD4 (membrane)-ABI. The HEK 293 cells were pre-transfected with the localization construct (Brg/Numb/CD4-ABI) and GFP-PYL reporter construct for 24 hours. 100 μM of ABA was then added to the cell culture and cells were observed by confocal microscopy for GFP expression after another 24 h.

FIG. 9 illustrates the ABA induced cell membrane localization of SOS can induce the MAP kinase signaling pathway as shown by an ABA-induced increase in phospho-Erk level. HEK 293T cells were co-transfected with myrystoylated SOS (myr-SOS) (a constitutive active form) only, or with myr-ABI and either SOS-PYL or SOS-FKBP3 (a negative control). 200 μM ABA was added after 24 hours and cells were collected at the indicated times to probe and quantify the phosphorylated form of Erk protein.

FIG. 10 provides possible structure of ABA antagonists.

FIG. 11 provides maps of AAV (adeno associated virus) constructs of ABA inducible therapeutic protein expression for use in gene therapy applications.

FIG. 12. The use of ABA-induced proximity for domain reconstitution, detected as induction of gene expression. (A) Dose response of ABA-induced luciferase activation for 24 hours in TC1 ES cells. (B) Dose response of ABA- or Rap-induced luciferase activation for 24 hours in CHO cells. (C) Time course (0 to 24 hours) of luciferase activation by ABA or Rap in E14 cells. (D) Time course (0 to 3 hours) of luciferase activation by ABA or Rap in E14 cells. (E) Time course of luciferase activity upon drug withdrawal after induction for 24 hours in CHO cells. The cells were transfected with the ABA- or Rap-activator cassette and the luciferase reporter for 24 hours before addition of ABA. For all experiments, the induction fold change was calculated relative to the values of noninduced samples. Data are the means±SEM (n W 3) from experiments of three independent transfections and induction from the same passage of cells, Independent experiments were repeated six or more times.

FIG. 13 illustrates the use of ABA-induced proximity to control protein subcellular localization and signal transduction. (A) Left: cytoplasmic localization of GFP-PYLcs to Numb-ABIcs induced by ABA in 293T cells. Numb-ABIcs was detected with an antibody that recognizes the FLAG tag. Right: nuclear localization of Numb-GFP-PYLcs to ABIcs-NLS induced by ABA in 293T cells. In both panels, nuclei are stained with DAPI. (B) ABA-induced ERK phosphorylation by SOS localization. Left panel: Cells were transfected with myr-ABIcs and SOS-PYLcs (lanes 1 to 5), myr-ABIcs and SOS-FKBP (lanes 6 and 7), or SOS-PYLcs only (lanes 8 and 9) for 24 hours, and then 250 ABA was added for the indicated period. Right: quantification of ERK phosphorylation in the left panel. The fold change was calculated by a-phos ERK/a-HA (for SOS fusion proteins) and then normalized to lane 1. The results are representative of three independent experiments. a-phos ERK, an antibody that recognizes phosphorylated ERK1/2; a-HA, an antibody that recognizes the HA tag; a-FLAG, an antibody that recognizes the FLAG tag; a-Hsp90, an antibody that recognizes heat shock protein 90; a-ERK, an antibody that recognizes total ERK1/2.

FIG. 14 illustrates that the ABA and Rap systems independent control transcription and protein localization. (A) Independent induction of luciferase by ABA and Rap for 24 hours in CHO cells. The cells were transfected with the ABA- or Rap-activator cassette and the luciferase reporter for 24 hours before addition of drug. Induction fold change was calculated relative to the values of noninduced samples. Data are the means±SEM (n=3) from experiments of three independent transfections and induction from the same passage of cells. Six independent repeats were performed. (B) Independent localization of mCherry-PYLcs and GFP-Frb by ABA and Rap. 293T cells were transfected with all of the indicated constructs 24 hours before the addition of ABA, Rap, or both at the indicated concentrations for 2 hours.

FIG. 15 illustrates that ABA-induced proximity can be engineered free of phosphatase activity. (A) Sequence alignment of the PP2C domain from P. tetraurelia (SEQ ID NO:1), A. thaliana (SEQ ID NO:2), M. mulatta (SEQ ID NO:3), H. Sapiens (SEQ ID NO:4), G. gallus (SEQ ID NO:5), C. elegans (SEQ ID NO:6), D. rerio (SEQ ID NO:7), M. musculus (SEQ ID NO:8), R. norvegicus (SEQ ID NO:9), and S. pombe (SEQ ID NO:10). Abbreviations for the amino acids are as follows: A, Ala; C, Cys; D, Asp; E, Glu; F, Phe; G, Gly; H, His; I, Ile; K, Lys: L, Leu; M, Met; N, Asn; P. Pro; Q, Gln; R, Arg; 3, Ser; T, Thr; V, Val; W, Trp; and Y, Tyr. (B) Phosphatase activity of wild-type and mutant GST-ABIcs. Phosphatase activity was measured by the ProFluor Ser/Thr PPase Assay Kit (20). Data are the means±SEM (n=3) of three independent experiments. (C) Pull-down of GFP-PYLcs from whole-cell lysates by wild-type (WT) or mutant GST-ABIcs. GFP-PYLcs and GST-ABIcs were detected by antibodies against GFP (a-GFP) or GST (a-GST), Data shown are representative of three independent experiments. (D) Luciferase activation by ABA in CHO cells expressing VP16-PYLcs and wild-type or mutant Gal4 DBD-ABIcs transgenes for 24 hours. Data are the means±SEM from experiments of three independent transfections and induction from the same passage of cells.

FIG. 16 illustrates that ABA is stable and orally available. CHO cells were transfected with the ABA activator cassette and luciferase reporter for 24 hours before addition of ABA-containing serum. (A) ABA stability in cell culture. ABA (100 μM) was incubated with CHO cells for the indicated times and then the medium was used for luciferase activation, (B) Biofunctional assay used to evaluate ABA concentration in serum. (C) ABA stability in human serum. ABA (1 mM) was incubated with fresh human serum for the indicated times and then used [10% (v/v) of culture medium] for luciferase activation. (D) Oral availability of ABA in mice. Serum was collected from mice at the indicated times after gavage of ABA in a mixture of ethanol, Tween 20, and Cremophor (4:3:1), and then 10% (v/v) of culture medium was added and applied to cells for luciferase activation. Induction fold change was calculated on the basis of the values of cells that were not exposed to serum. The data in (A) and (C) are the means±SEM of triplicates from representative experiments; the data in (D) are the means±SEM (n=6 to 8).

FIG. 17 illustrates the crystal structure of ABI1-(+)-ABA-PYL1 complex. Green, ABI1; yellow, PYL1; purple, gate-and-latch loop of PYL1; cyan, (+)-ABA. This image was made with VMD. VMD is developed with NH support by the Theoretical and Computational Biophysics group at the Beckman Institute, University of Illinois at Urbana-Champaign. (Miyazono, K. et al. Structural basis of abscisic acid signalling. Nature 462, 609-614 (2009)).

FIG. 18 illustrates the dose response of ABA-induced luciferase activation in cultured mammalian cell lines, (A) NIH3T3 and (B) HEK 293T cells were transfected with ABA-activator cassette and luciferase reporter for 24 hours before adding ABA. Luciferase activity was measured 24 hours after ABA addition. Induction fold change was calculated relative to the values of noninduced samples. Data were the mean±SEM (n=3) from experiments of three independent transfections and induction from the same passage of cells. 6 independent experiments were repeated.

FIG. 19 illustrates the time course of luciferase activation by ABA or Rap in CHO cells, CHO cells were transfected with ABA or Rap activator cassette and luciferase reporter for 24 hours before adding ABA. Luciferase activity was measured from (A) 0 to 48 hours (B) 0 to 5 hours at the indicated time point after ABA addition. Induction fold change was calculated relative to the values of non-induced cells. Data were the mean±SEM from experiments of 3 independent transfections and induction from the same passage of cells, 3-6 independent experiments were repeated.

FIG. 20 illustrates the reversibility of ABA- and Rap-induced luciferase production in E14 cells under different washing conditions. E14 ES cells were transfected with (A) ABA- or (B) Rap activator cassette and luciferase reporter for 24 hours, and then added ABA or Rap for another 24 hours. Cells were then washed with (i) no wash, (ii) washed 3 times with fresh media without drug and incubated at 37° C. for 5 minutes between each wash (Short Wash), (iii) the same as (ii) but changed fresh media without drug every hour up to 5 hours (Long Wash). (iv) the same as (ii) but added FK506 in cash wash and after-wash incubation (Wash w/FK506). Induction fold change was calculated relative to the values of non-induced cells. Data were the mean±SEM from experiments of 3 independent transfections and induction from the same passage of cells.

FIG. 21 illustrates the amounts of fusion protein production in ABA and Rap system. CHO cells and E14 cells were transfected with ABA- or Rap-activator cassette for 48 hours before harvest. Whole cell lysates were blotted with anti-Gal4 DBD and anti-GAPDH antibody. Relative ratios of Gal4 DBD-fusion proteins in Rap and ABA systems were calculated by first normalizing Gal4 DBD to GAPDH and then relative to ABA system. Western bot and subsequent quantification was representative of 2 independent experiments.

FIG. 22 illustrates luciferase induction by ABA in different cell types. Cells were transfected with ABA-activator cassette and luciferase reporter for 24 hours before adding ABA. Luciferase activity was measured 24 hours after ABA (100 μM) addition. Induction fold change was calculated relative to the values of noninduced samples. Data were the mean±SEM of triplicates from representative experiments. CHO, Chinese hamster ovary cell line; NIH3T3, mouse fibroblast cell line; HEK293T, human embryonic kidney fibroblast cell line; COS7, monkey epithelial cell line; B35, rat neuronal cell line; Jurkat, human T cell line; E14, mouse embryonic stem cell line; TC1, mouse embryonic stem cell line; MEF, mouse embryonic fibroblasts

FIG. 23 illustrates the distribution of GFP-PYLcs with or without ABA in HEK 293T cells. Cells were transfected with GFP-PYLcs construct for 24 hours before adding ABA, Nuclei were stained with DAPI and GFP-PYL was monitored 2 hours after adding ABA. The quantification ratio of nuclear to cytoplasmic MR (mean fluorescence intensity) was measured and calculated as described in “Materials and Methods” section.

FIG. 24 illustrates the quantification of cytoplasmic localization of GFP-PYLcs, HEK293T cells were transfected with the GFP-PYLcs and Numb-ABIcs constructs (FIG. 2A) for the fusion proteins for 24 hours before adding ABA. Nuclei were stained with DAPI and cells were processed for imaging 2 hours after adding ABA. The quantification ratio of cytoplasmic to nuclear MA was measured and calculated as described in “Materials and Methods” section. Data were the mean±SEM (n=20).

FIG. 25 illustrates the ABA-induced nuclear localization of GFP-PYLcs. Nuclear localization of GFP-PYLcs to (A) Brg-ABIcs or (B) ABIc-NLS induced by ABA in HEK 293T cells. Brg-ABIcs was stained by anti-FLAG antibody. ABIc included PP2C domain and entire C terminal of ABI1. Cells were transfected with the indicated constructs for the fusion proteins for 24 hours before adding ABA. Nuclei were stained with DAPI and cells were processed for imaging 2 hours after adding ABA. The quantification ratio of nuclear to cytoplasmic MFI was measured and calculated as described in “Materials and Methods” section, Data were the mean±SEM (n=20).

FIG. 26 illustrates the ABA-induced membrane localization of GFP-PYLcs. Membrane localization of GFP-PYLcs to (A) CD4-ABIcs or (B) myr-ABIcs induced by ABA in 293T cells. CD4-ABIcs and myr-ABIcs were stained by anti-FLAG antibody. The myr sequence also targets the endoplasmic reticulum (ER), mitochondria, and other intracellular membranes, whereas CD4 directs localization to the cell membrane. Cells were transfected with the indicated constructs for the fusion proteins for 24 hours before adding ABA. Nuclei were stained with DAPI and cells were processed for imaging 2 hours after adding ABA.

FIG. 27 illustrates the ABA-induced nuclear localization of Numb-GFP-PYLcs. (A) Numb-GFP-PYLcs only, or (B) Numb-GFP-PYLcs co-transfected with ABIc-NLS was incubated with 450 μM ABA in HEK 293T cells. ABIc included PP2C domain and entire C-terminal of ABI1 Numb-GFP-PYLcs was localized to the nucleus in the presence of ABIc-NLS upon ABA addition. The degree of Numb-GFP-PYLcs nuclear localization varied among cells as shown in the second and third rows of panel B. Cells were transfected with the indicated constructs for the fusion proteins for 24 hours before adding ABA. Nuclei were stained with DAPI and cells were processed for imaging 2 hours after adding ABA. The quantification ratio of nuclear to cytoplasmic MFI was measured and calculated as described in “Materials and Methods” section. “1” is Numb-GFP-PYL, no ABA; “2” is Numb-GFP-PYL, with ABA; “3” is Numb-GFP-PYL plus NLS-ABIcs, no ABA; “4” is Numb-GFP-PYL plus NLS-ABIcs, with ABA. Data were the mean±SEM (n=20). Cell population quantification was performed by counting 300 cells in each condition from each experiment for no localization (No Nuclear: no clear visible GFP in nucleus) or localization (Nuclear: clear visible nuclear GFP). Data were the mean±SEM from 3 independent transfections.

FIG. 28 illustrates orthogonal induction of luciferase by ABA and Rap in NH 3T3 cells, Cells were transfected with ABA- or Rap-activator cassette and luciferase reporter for 24 hours before adding ABA. Luciferase activity was measured 24 hours after ABA addition. Data were the mean±SEM (n=3) from experiments of three independent transfections and induction from the same passage of cells, 6 independent experiments were repeated.

FIG. 29 illustrates the independent ABA- or Rap-induced membrane localization of GFP fusion proteins. Independent membrane localization of (A) GFP-PYLcs to myr-ABIcs or (B) GFP-Erb to myr-FKBP by ABA or Rap, respectively. GFP-PYLcs done are homogenously distributed (FIG. S7.). Cells were transfected with the indicated constructs for the fusion proteins for 24 hours before adding ABA or Rap. Nuclei were stained with DAPI and cells were processed for imaging 2 hours after adding the inducers.

FIG. 30 illustrates the independent relocalization of GFP and mCherry fusion proteins by ABA and Rap systems. Independent nuclear localization of mCherry-PYLcs to Brg-ABIcs by ABA or membrane localization of GFP-Frb to CD4-FKBP by Rap, respectively. Cells were transfected with the indicated constructs for the fusion proteins for 24 hours before adding ABA or Rap, or both. Nuclei were stained with DAPI and cells were processed for imaging 2 hours after adding the inducers.

FIG. 31 provides quantification of GFP-PYLcs pull-down by wild-type or mutant ABI. Quantification was calculated by normalizing to wild-type GST-ABIcs pull-down. Data were the mean±SEM of 3 independent experiments.

FIG. 32 illustrates ABA stability in FBS. ABA (1 mM) was incubated with heat-inactivated FBS for indicated time and then used (added 10% v/v of culture media) for luciferase activation. The data were the mean±SEM of triplicates from representative experiments. Experiments were repeated 3 times.

FIG. 33 illustrates bioavailability of ABA in mice. Serum was collected from mice at the indicated times after intraperitoneal (IP) injection of ABA (100 mg/kg mouse weight) in a 1:9 mixture of ethanol and PBS, and then used (added 10% v/v of culture media) for luciferase activation. Induction fold-change was calculated based on the values of noninduced cells. The data were the mean±SEM (n=4-6).

FIG. 34 illustrates the effect of ABA on lymphocyte proliferation. Untouched T cells were isolated from spleens of C57BL/6J mice using MACS T cell isolation kit (Miltenyi Biotec). Isolated T cells were stimulated for 72 hours at 37° C. with plate bound monoclonal antibodies against CD3 (10 μg/ml) and CD28 (2 μg/ml) prior to cell count measurements. Mean cell counts±SEM were obtained from 2 independent anti-CD3 antibody and anti-CD28 antibody stimulations of untouched T cells isolated from 3 mice.

### DETAILED DESCRIPTION

Methods of inducing proximity of chimeric molecules in a cell are provided. Aspects of the methods include contacting a cell with an amount of an alkenyl substituted cycloaliphatic (ASC) inducer compound, e.g., abscisic acid, effective to induce proximity of first and second chimeric molecules. Also provided are compositions and kits for practicing various embodiments of the methods. Methods of the invention find use in a variety of different applications, including transcription induction applications.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed, to the extent that such combinations embrace operable processes and/or devices/systems/kits. In addition, all sub-combinations listed in the embodiments describing such variables are also specifically embraced by the present invention and are disclosed herein just as if each and every such sub-combination of chemical groups was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing aspects of various embodiments of the invention, aspects of embodiments of the methods will be described first in greater detail. Next, examples of applications in which the methods find use are described in greater detail. Following this discussion, a description of kits that may be used in practicing embodiments of the methods is provided.

### Methods

As summarized above, aspects of the invention include inducing proximity of at least first and second chimeric molecules. By “inducing proximity” is meant that two or more, such as three or more, including four or more, chimeric molecules are spatially associated with each other through a binding event mediated by the alkenyl substituted cycloaliphatic (ASC) inducer compound. Spatial association is characterized by the presence of a binding complex that includes the inducer and the at least first and second chimeric molecules. In the binding complex, each member or component is bound to at least one other member of the complex. In this binding complex, binding amongst the various components may vary. For example, the inducer may mediate a direct binding event between domains of first and second chimeric molecules that would not occur in the absence of the inducer. For example, in the presence of the inducer, a domain of a first chimeric molecule may bind to a domain of a second chimeric molecule, where this binding event would not occur in the absence of the inducer. In other instances, the inducer may simultaneously bind to domains of the first and second chimeric molecules, thereby producing the binding complex and desired spatial association. Inducing proximity may encompass spatial association (i.e. binding complex formation), of identical or different chimeric molecules. Depending on whether the chimeric molecules are the same or different, proximity induction may be viewed as homodimerization, homo-oligomerization, hetero-dimerization and hetero-oligomerization. Homo-dimerization and homo-oligomerization refer to the association of like components (i.e., identical chimeric proteins) to form dimers or oligomers. Hetero-dimerization and hetero-oligomerization refer to the association of dissimilar chimeric molecules (e.g., chimeric molecules that include different effector domains) to form dimers or oligomers. “Oligomerization”, “oligomerize” and “oligomer”, as the terms are used herein, with or without prefixes, are intended to encompass “dimerization”, “dimerize” and “dimer”, absent an explicit indication to the contrary. In some instances, the ASC inducer compound induces proximity of the first and second chimeric molecules, where first and chimeric molecules bind directly to each other in the presence of the ASC inducer compound but not in the absence of the ASC inducer compound.

Before providing additional details regarding various aspects of the methods, additional details regarding aspects of embodiments of ASC inducer compounds and the chimeric molecules finding use in methods of invention are provided.

### ASC Inducer Compounds

As summarized above, aspects of the invention include mulitimerization of chimeric molecules with an ASC inducer compound. ASC inducer compounds of the invention are small molecules and are non-toxic. By small molecule is meant a molecule having a molecular weight of 5000 daltons or less, such as 2500 daltons or less, including 1000 daltons or less, e.g., 500 daltons or less. By non-toxic is meant that the inducers exhibit substantially no, if any, toxicity at concentrations of 1 g or more/kg body weight, such as 2.5 g or more/kg body weight, including 5 g or more/kg body weight.

In certain embodiments, an ASC inducer compound of the invention includes a cycloaliphatic ring substituted with an alkenyl group. In certain embodiments, the cycloaliphatic ring is further substituted with a hydroxyl and/or oxo group. In certain embodiments, the carbon of the cycloaliphatic ring that is substituted with the alkenyl group is further substituted with a hydroxyl group. In certain embodiments, the cycloaliphatic ring system is an analog of a cyclohex-2-enone ring system.

In certain embodiments, an alkenyl substituted cycloaliphatic compound of the invention includes a cyclohexene or a cyclohexane ring, such as is found in a cyclohexenone group (e.g. a cyclohex-2-enone), a cyclohexanone group, a hydroxy-cyclohexane group, a hydroxy-cyclohexene group (e.g., a cyclohex-2-enol group) or a methylenecyclohexane group (e.g. a 3-methylenecyclohexan-1-ol group); where the cycloaliphatic ring is substituted with an alkenyl group of about 2 to 20 carbons in length, that includes 1, 2, 3, 4, 5, 6, 7, 8, 9 or 10 unsaturated bonds. In some embodiments, the alkenyl substituent includes a conjugated series of unsaturated bonds. In some embodiments, the alkenyl substituent is 4 carbons in length and includes 2 conjugated double bonds. In particular embodiments, the alkenyl substituent is conjugated to the cycloaliphatic ring system.

In certain embodiments, the terminal position of the alkenyl substituent is further substituted with a carboxy, hydroxy, an aldehyde, an ester or an amido group.

In certain embodiments, ASC inducers of the invention include a cycloaliphatic ring, linked via an alkenyl substituent to a carboxy, hydroxy, an aldehyde, an ester or an amido group.

In certain embodiments, an alkenyl substituted cycloaliphatic compound of the invention includes:

a cycloaliphatic ring system selected from a cyclohexenone, a cyclohexanone, a cyclohexane or hydroxy-cyclohexane, a hydroxy-cyclohexene and a methylenecyclohexanol or methylenecyclohexane;

a terminal group selected from hydrogen, a carbonyl, an amido, an alkoxylcarbonyl, carboxy, hydroxy and formyl; and

an alkenyl linking group that links said cycloaliphatic ring system to said terminal group.

In certain embodiments, a ASC inducers of the invention are described by formula (I):

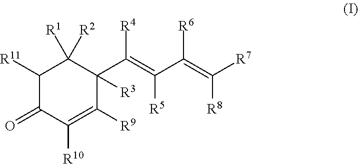

where R¹, R², R³, R⁴, R⁵, R⁶, R⁷, R⁸, R⁹, R¹⁰ and R¹¹ are independently selected from hydrogen, an alkyl, an aryl, an alkenyl, an alkynyl, a carbonyl, an acyl, a halogen, a hydroxy, an alkoxy, an aryloxy, a heterocyclic group, where optionally any two of R¹, R², R³, R⁴, R⁵, R⁶, R⁷, R⁸, R⁹, R¹⁰ and R¹¹ can be cyclically linked.

In some embodiments, in formula (I), R¹, R², R⁴, R⁵, R⁶, R⁸, R⁹, R¹⁰ and R¹¹ are independently selected from hydrogen, an alkyl and a halogen (e.g., fluoro).

In some embodiments, in formula (I), R³ is selected from hydrogen, hydroxy, and an alkoxy.

In some embodiments, in formula (I), R⁷ is selected from hydrogen, a carbonyl (e.g., a carboxy or a formyl), a hydroxymethyl and an alkenyl (e.g., —CH═CHCOCH₃).

In some embodiments, in formula (I), R⁶ and R⁸ are cyclically linked (e.g., to form a cyclopentene ring).

In some embodiments, a compound of the invention is of the structure of formula (II):

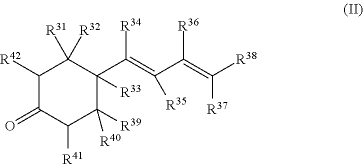

where R³¹, R³², R³³, R³⁴, R³⁵, R³⁶, R³⁷, R³⁸, R³⁹, R⁴⁰, R⁴¹ and R⁴² are independently selected from hydrogen, an alkyl, an aryl, an alkenyl, an alkynyl, a carbonyl, an acyl, a halogen, a hydroxy, an alkoxy, an aryloxy, a heterocyclic group, where optionally any two of R³¹, R³², R³³, R³⁴, R³⁵, R³⁶, R³⁷, R³⁸, R³⁹, R⁴⁰, R⁴¹ and R⁴² can be cyclically linked.

In some embodiments, in formula (II), R³¹, R³², R³⁴, R³⁵, R³⁶, R³⁷, R³⁹, R⁴⁰, R⁴¹ and R⁴² are independently selected from hydrogen, an alkyl and a halogen (e.g., fluoro).

In some embodiments, in formula (II), R³⁸ is selected from hydrogen, carboxy, an ester, a hydroxymethyl, formyl and an alkenyl (e.g., —CH═CHCOCH₃).

In some embodiments, in formula (II), R³³ is selected from hydrogen, hydroxy, and an alkoxy.

In some embodiments, in formula (II), R³⁶ and R³⁷ are cyclically linked (e.g., to form a cyclopentene ring).

In some embodiments, a compound of the invention is of the structure of formula (III):

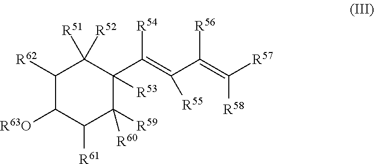

where R⁵¹, R⁵², R⁵³, R⁵⁴, R⁵⁵, R⁵⁶, R⁵⁷, R⁵⁸, R⁵⁹, R⁶⁰, R⁶¹, R⁶² and R⁶³ are independently selected from hydrogen, an alkyl, an aryl, an alkenyl, an alkynyl, a carbonyl, an acyl, a halogen, a hydroxy, an alkoxy, an aryloxy, a heterocyclic group, where optionally any two of R⁵¹, R⁵², R⁵³, R⁵⁴, R⁵⁵, R⁵⁶, R⁵⁷, R⁵⁸, R⁵⁹, R⁶⁰, R⁶¹, R⁶² and R⁶³ can be cyclically linked.

In some embodiments, in formula (III), R⁵¹, R⁵², R⁵⁴, R⁵⁵, R⁵⁶, R⁵⁸, R⁵⁹, R⁶⁰, R⁶¹ and R⁶² are independently selected from hydrogen, an alkyl and a halogen (e.g., fluoro).

In some embodiments, in formula (III), R⁵⁷ is selected from hydrogen, carboxy, an ester, a hydroxymethyl, formyl and an alkenyl (e.g., —CH═CHCOCH₃).

In some embodiments, in formula (III), R⁵³ is selected from hydrogen, hydroxy, and an alkoxy.

In some embodiments, in formula (III), R⁶³ is selected from hydrogen, an alkyl, and an acyl.

In some embodiments, in formula (III), R⁵⁶ and R⁵⁸ are cyclically linked (e.g., to form a cyclopentene ring).

In some embodiments, in formula (III), R⁵³ and R⁵⁹ are cyclically linked (e.g., to form an epoxide).

In some embodiments, in formula (III), R⁵² and R⁵⁹ are cyclically linked (e.g., to form an ether bridge).

In certain embodiments, a compound of the invention is of the structure of formula (IV):

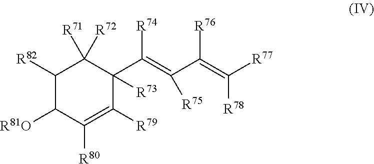

where R⁷¹, R⁷², R⁷³, R⁷⁴, R⁷⁵, R⁷⁶, R⁷⁷, R⁷⁸, R⁷⁹, R⁸⁰, R⁸¹ and R⁸² are independently selected from hydrogen, an alkyl, an aryl, an alkenyl, an alkynyl, a carbonyl, an acyl, a halogen, a hydroxy, an alkoxy, an aryloxy, a heterocyclic group, where optionally any two of R⁷¹, R⁷², R⁷³, R⁷⁴, R⁷⁵, R⁷⁶, R⁷⁷, R⁷⁸, R⁷⁹, R⁸⁰, R⁸¹ and R⁸² can be cyclically linked.

In particular embodiments, in formula (IV), R⁷¹, R⁷², R⁷⁴, R⁷⁵, R⁷⁶, R⁷⁸, R⁷⁹, R⁸⁰ and R⁸² are independently selected from hydrogen, an alkyl and a halogen (e.g., fluoro).

In particular embodiments, in formula (IV), R⁷⁷ is selected from hydrogen, carboxy, an ester, a hydroxymethyl, formyl and an alkenyl (e.g., —CH═CHCOCH₃).

In particular embodiments, in formula (IV), R⁷³ is selected from hydrogen, hydroxy, and an alkoxy.

In particular embodiments, in formula (IV), R⁸¹ is selected from hydrogen, an alkyl, and an acyl.

In particular embodiments, in formula (IV), R⁷⁶ and R⁷⁸ are cyclically linked (e.g., to form a cyclopentene ring).

In certain embodiments, a compound of the invention is of the structure of formula (V):

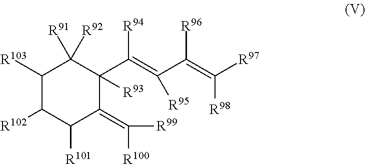

where R⁹¹, R⁹², R⁹³, R⁹⁴, R⁹⁵, R⁹⁶, R⁹⁷, R⁹⁸, R⁹⁹, R¹⁰⁰, R¹⁰¹, R¹⁰² and R¹⁰³ are independently selected from hydrogen, an alkyl, an aryl, an alkenyl, an alkynyl, a carbonyl, an acyl, a halogen, a hydroxy, an alkoxy, an aryloxy, a heterocyclic group, where optionally any two of R⁹¹, R⁹², R⁹³, R⁹⁴, R⁹⁵, R⁹⁶, R⁹⁷, R⁹⁸, R⁹⁹, R¹⁰⁰, R¹⁰¹, R¹⁰² and R¹⁰³ can be cyclically linked.

In particular embodiments, in formula (V), R⁹¹, R⁹², R⁹⁴, R⁹⁵, R⁹⁶, R⁹⁸, R¹⁰¹ and R¹⁰³ are independently selected from hydrogen, an alkyl and a halogen (e.g., fluoro).

In particular embodiments, in formula (V), R⁹⁷ is selected from hydrogen, carboxy, an ester, a hydroxymethyl, formyl and an alkenyl (e.g., —CH═CHCOCH₃).

In particular embodiments, in formula (V), R⁹³ is selected from hydrogen, hydroxy, and an alkoxy.

In particular embodiments, in formula (V), R⁹⁶ and R⁹⁸ are cyclically linked (e.g., to form a cyclopentene ring).

In particular embodiments, in formula (V), R⁹⁹ and R¹⁰⁰ are independently selected from hydrogen and an alkyl.

In particular embodiments, in formula (V), R¹⁰² is selected from hydrogen, hydroxyl, an acyloxy and an alkoxy.

In certain embodiments, a compound of the invention is of the structure of formula (VI):

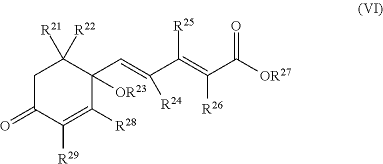

where R²¹, R²², R²³, R²⁴, R²⁵, R²⁶, R²⁷, R²⁸ and R²⁹ are independently selected from hydrogen, a halogen (e.g., fluoro), an alkyl (e.g., a lower alkyl or a hydroxymethylene), an alkoxy, a hydroxy, an alkynyl, an alkenyl, a hydroxymethyl, a trifluoromethyl; where optionally any two of adjacent where R²¹, R²², R²³, R²⁴, R²⁵, R²⁶, R²⁷, R²⁸ and R²⁹ can be cyclically linked

In particular embodiments, in formula (VI), R²¹, R²², R²⁴, R²⁵, R²⁶, R²⁸ and R²⁹ are independently selected from hydrogen, an alkyl and a halogen (e.g., fluoro); and R²³ and R²⁷ are independently selected from hydrogen and an alkyl.

In particular embodiments, in formula (VI), R²¹ and R²² are independently selected from hydrogen, a lower alkyl (e.g., a methyl, a hydroxymethyl, a trifluoromethyl, a difluoromethyl), a halogen (e.g., a fluoro), hydroxy, an alkoxy, an alkenyl, and an alkynyl (e.g., an acetylenyl, —CCH).

In particular embodiments, in formula (VI), R²³ is selected from hydrogen and a lower alkyl (e.g., a methyl).

In particular embodiments, in formula (VI), R²⁴ is hydrogen.

In particular embodiments, in formula (VI), R²⁵ is selected from hydrogen, a lower alkyl (e.g., a methyl or a hydroxymethyl) and a halogen (e.g., a fluoro).

In particular embodiments, in formula (VI), R²⁶ is selected from hydrogen and a halogen (e.g., fluoro).

In particular embodiments, in formula (VI), R²⁷ is selected from hydrogen and a lower alkyl (e.g., a methyl or an ethyl).

In particular embodiments, in formula (VI), R²⁸ is selected from hydrogen, a lower alkyl (e.g., a methyl, an ethyl or a hydroxymethyl).

In particular embodiments, in formula (VI), R²⁹ is selected from hydrogen and a halogen (e.g., fluoro, chloro, bromo or iodo).

In particular embodiments, in formula (VI), R²⁷ is selected from hydrogen

In certain embodiments, a compound of the invention is of the following structure:

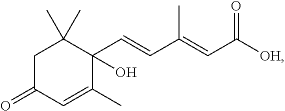 [SMILES: CC1=CC(=O)CC(C)(C)C1(O)/C=C/C(C)=C/C(=O)O]

or a substituted derivative thereof.

In particular embodiments, a compound of the invention is of the following structure:

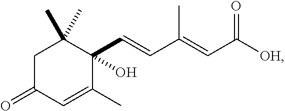

or a substituted derivative thereof.

Specific ASC inducer compounds of interest include, but are not limited to: abscisic acid, abscisic aldehyde, abscisic alcohol, methyl abscisate, ethyl abscisate, xanthoxin, phaseic acid, dihydrophaseic acid, epi-dihydrophaseic acid, methyl phaseate, ethyl phaseate, alpha-ionone, beta-ionone, damascene, beta-damascenone, 4′-oxo-alpha-ionylideneacetic acid, 4′-hydroxy-alpha-ionylideneacetic acid, alpha-ionylideneacetic acid, epoxy-beta-ionylideneacetic acid, 2′,3′-dihydroabscisic acid, 7′-hydroxyabscisic acid, 8′-hydroxyabscisic acid, 8′-hydroxy-2′,3′-dihydroabscisic acid, and substituted derivatives thereof.

### Chimeric Molecules

As reviewed above, the ASC inducer compounds are employed to induce proximity of chimeric molecules. Chimeric molecules whose proximity is induced by ASC inducer compounds in accordance with embodiments of the invention are molecules that include at least two distinct heterologous domains which are stably associated with each other. By “heterologous”, it is meant that the at least two distinct domains do not naturally occur in the same molecule. As such, the chimeric molecules are composed of at least two distinct domains of different origin. As the two domains of the chimeric molecules are stably associated with each other, they do not dissociate from other under cellular conditions, e.g., conditions at the surface of a cell, conditions inside of a cell, etc. The two domains may be associated with each other via covalent or non-covalent binding, as desired. In principle, the chimeric molecules may be any molecule that can be provided in a cell, and may be nucleic acids, proteins, or composites thereof, e.g., nucleic acid/peptide composite molecules. In some instances, the chimeric molecules are chimeric proteins (i.e., fusion proteins), that include at least the two distinct domains, i.e., an ASC inducer compound specific binding domain and an effector domain.

Each chimeric molecule whose proximity is induced by the ASC inducer compounds includes at least a first ASC inducer domain and an effector domain. The ASC inducer domain is a domain which participates in some manner in the ASC inducer-mediated binding event that results in the desired proximity induction of the at least first and second chimeric molecules. As such, ASC inducer domains are domains that participate in the binding complex that characterizes the proximity induction, e.g., as described above. In some instances, these ASC inducer domains bind directly to each other when in the presence of the ASC inducer, but not in the absence of the ASC inducer. In some instances, the ASC inducer domains simultaneously specifically bind to the ASC inducer compound.

In some instances, the ASC inducer domain specifically binds to the ASC inducer compound and is therefore an ASC inducer compound binding domain. The terms “specific binding,” “specifically bind,” and the like, refer to the ability of the ASC inducer compound binding domain to preferentially bind directly to the ASC inducer relative to other molecules or moieties in the cell. In certain embodiments, the affinity between a binding domain and the ASC inducer compound to which it specifically binds when they are specifically bound to each other in a binding complex is characterized by a K_{D} (dissociation constant) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. ASC inducer specific binding domains may vary widely.

In some instances, one of the chimeric molecules, e.g., the first chimeric molecule, includes an ASC inducer compound specific binding domain as the ASC inducer domain. In these embodiments, this ASC inducer domain is a domain that specifically binds to abscisic acid. ASC inducer compound specific binding domains of interest include, but are not limited to: the abscisic acid binding domains of the pyrabactin resistance (PYR)/PYR1-like (PYL)/regulatory component of ABA receptor (RCAR) family of intracellular proteins. The PYR/PYL/RCAR abscisic acid binding domains are those domains or regions of PYR/PYL/RCAR proteins, (e.g., pyrabactin resistance 1, PYR1-Like proteins, etc.) that specifically bind to abscisic acid. Accordingly, ASC inducer binding domains include a full length PYR1 or PYL proteins (e.g., PYL1, PYL 2, PYL 3, PYL 4, PYL 5, PYL 6, PYL, PYL 8, PYL 9, PYL 10, PYL11, PYL12, PYL13), as well as portions or mutants thereof that bind to abscisic acid, e.g., amino acid residues 33-209 of PYL1 from Arabidopsis thaliana (SEQ ID NO:11).

As mentioned above, ASC inducer domains also include domains that bind to ASC domains of other chimeric molecules (e.g., PYR/PYL abscisic acid binding domains), in the presence of the ASC inducer compound but not in the absence of the ASC inducer compound. Examples of such ASC inducer domains include PP2C inducer domains. The PP2C inducer domains are those PYR/PYL binding domains found in group A type 2 C protein phosphatases (PP2Cs), where PP2Cs have PYL(+ABA) binding domains. Accordingly, ASC inducer domains include the full length PP2C proteins (e.g., ABI1), as well as portions or mutants thereof that bind to abscisic acid, e.g., amino acid residues 126-423 of ABI1 from Arabidopsis thaliana (SEQ ID NO:12). In some instances, the PP2C ASC inducer domain is a phosphatase negative mutant, e.g., a mutant of PP2C that retains its ability to specifically bind to PYR/PYL (+ABA) and yet has reduced if not absent phosphatase activity. An example of such a phosphatase negative PP2C ASC inducer domain is the ABI1 D143A mutant described in the Experimental Section, below.

In addition to the ASC inducer specific binding domain, the chimeric molecules also include an effector domain. The effector domain is a domain that can modulate in a desirable way a physiological action or cellular process, i.e., cause a cellular activity, as a result of proximity induction of the chimeric molecules. Causing a cellular activity of interest includes both activating a process and inhibiting a process of interest. For example, proximity induction may result in the activation of a process such as a signaling cascade or transcription event. Proximity induction may result in inhibition of a process, such as a signaling cascade or transcription event. The precise mechanism of how the cellular process is modulated by the ASC induced proximity of chimeric molecules may vary, where examples include re-locating cellular proteins to specific locations or compartments to activate a process of interest (e.g., relocating SOS to the cell membrane to activate the ras pathway) or block a cellular process (e.g., directing a transcription factor (such as NFAT) out of nucleus), etc. As such, proximity induction may bring the effector domains of two or more chimeric molecules into close proximity with one another thus triggering cellular processes normally associated with close proximity of the respective effector domains, such as initiation of transcription, signal transduction, etc. Cellular processes which can be triggered by ASC inducer compound mediated proximity include a change in state, such as a physical state, e.g., conformational change, change in binding partner, cell death, initiation of transcription, channel opening, ion release, e.g., Ca⁺² etc., or change in a chemical state, such as a chemical reaction, e.g., acylation, methylation, hydrolysis, phosphorylation or dephosphorylation, change in redox state, protein stability, RNA splicing, protein splicing, rearrangement, or the like. Thus, any such process which can be triggered by ASC inducer compound mediated proximity may be effectuated by the effector domains of the chimeric molecules. Within a given set, e.g., pair, of chimeric molecules, the effector domains may vary with respect to the nature of the desired action to be caused by ASC-inducer compound mediated proximity. Effector domains of interest include, but are not limited to: effector domains whose ASC inducer compound mediated association induces a signal which results in a series of events resulting in transcriptional activation of one or more genes; effector domains whose ASC inducer compound mediated association induces initiation of transcription directly via complexation of the spatially associated chimeric molecules with a DNA transcriptional initiation region; effector domains whose ASC inducer compound mediated association results in exocytosis; effector domains whose ASC inducer compound mediated association induces bridging of one or more similar or dissimilar molecules or cells; effector domains whose ASC inducer compound mediated association induces destabilization and/or degradation or inactivation of the spatially associated chimeric molecules; effector domains whose associate directs the proximity induced chimeric molecules to a specific cellular location, e.g., out of the nucleus, into the nucleus, to the membrane, etc., e.g., to activate a cellular process, e.g., signaling pathway, or inhibit a cellular process, e.g., transcription. Effector domains may be selected from a wide variety of protein domains including DNA binding domains (e.g., a GAL4 or ZFHD1 DNA-binding domain), transcription activation domains (e.g., a VP16 or p65 transcription activation domain), cellular localization domains (e.g., domains that are capable of directing a proximity induced complex to a particular location of a cell, e.g., membrane, nucleus, etc.); and signaling domains (e.g., domains which are capable upon clustering or multimerization, of triggering cell growth, proliferation, differentiation, apoptosis, gene transcription, etc.). Specific examples of effector domains of interest include, but are not limited to, those described in published PCT application WO/1994/018317 and WO/1999/041258; where the specific effector domains disclosed in these published applications are herein incorporated by reference.

A given chimeric molecule may include a single ASC inducer domain or multiple copies of the domain, e.g., 2 or more, 3 or more, etc. Similarly, a given chimeric molecule may include a single effector domain or multiple copies of an effector domain, e.g., 2 or more, 3 or more, etc. Additional domains may be present in a given chimeric molecule, e.g., linker domains, subcellular targeting domains, etc.

### Target Cells

As summarized above, aspects of methods of invention include contacting a target cell with an ASC inducer compounds, where the cell includes at least first and second chimeric molecules, e.g., as described above, in a manner effective to multimerize the chimeric molecules and achieve a desired effect, such as cell signaling or transcription. The target cell that is contacted with the ASC inducer compound may vary depending on the specific application being performed. Target cells of interest include animal cells, where specific types of animal cells include, but are not limited to: insect, worm or mammalian cells. Various mammalian cells may be used, including, by way of example, equine, bovine, ovine, canine, feline, murine, non-human primate and human cells. Among the various species, various types of cells may be used, such as hematopoietic, neural, glial, mesenchymal, cutaneous, mucosal, stromal, muscle (including smooth muscle cells), spleen, reticulo-endothelial, epithelial, endothelial, hepatic, kidney, gastrointestinal, pulmonary, fibroblast, and other cell types. Hematopoietic cells of interest include any of the nucleated cells which may be involved with the erythroid, lymphoid or myelomonocytic lineages, as well as myoblasts and fibroblasts. Also of interest are stem and progenitor cells, such as hematopoietic, neural, stromal, muscle, hepatic, pulmonary, gastrointestinal and mesenchymal stem cells, such as ES cells, epi-ES cells and induced pluripotent stem cells (iPS cells).

As summarized above, the target cells that are contacted with the ASC inducer compounds include at least the first and second chimeric molecules. As such, the target cells are cells that have been engineered to include the first and second chimeric molecules. The protocol by which the cells are engineered to include the desired chimeric molecules may vary depending on one or more different considerations, such as the nature of the target cell, the nature of the chimeric molecules, etc.

Where the chimeric molecules are chimeric proteins, the cell may include expression constructs having coding sequences for the chimeric proteins under the control of a suitable promoter. The coding sequences will vary depending on the particular nature of the chimeric protein encoded thereby, and will include at least a first domain that encodes the ASC inducer domain and a second domain that encodes the effector domain. The two domains may be joined directly or linked to each other by a linking domain. The domains encoding the fusion protein are in operational combination, i.e., operably linked, with requisite transcriptional mediation or regulatory element(s). Requisite transcriptional mediation elements that may be present in the expression module include promoters (including tissue specific promoters), enhancers, termination and polyadenylation signal elements, splicing signal elements, and the like. Of interest in some instances are inducible expression systems.

Optionally the target cells may further contain a second recombinant genetic construct, or second series of such construct(s), containing a target gene under the transcriptional control of a transcriptional control element (e.g. promoter/enhancer) responsive to a signal triggered by ASC inducer compound mediated proximity of the chimeric proteins. These constructs are recombinant in the sense that the target gene is not naturally under the transcriptional control of the responsive transcriptional control element. In one aspect of the invention the DNA construct contains (a) a transcriptional control element responsive to the proximity of a chimeric protein as described above, and (b) flanking DNA sequence from a target gene permitting the homologous recombination of the transcriptional control element into a host cell in association with the target gene. In other embodiments the construct contains a desired gene and flanking DNA sequence from a target locus permitting the homologous recombination of the target gene into the desired locus. The construct may also contain the responsive transcriptional control element, or the responsive element may be provided by the locus. The target gene may encode a variety of different types of products, such as but not limited to: a surface membrane protein, a secreted protein, a cytoplasmic protein or a ribozyme or an antisense sequence.

The various expression constructs in the target may be chromosomally integrated or maintained episomally, as desired. Accordingly, in some instances the expression constructs are chromosomally integrated in a cell. Alternatively, one or more of the expression constructs may be episomally maintained, as desired.

The target cells may be prepared using any convenient protocol, where the protocol may vary depending on nature of the target cell, the location of the target cell, e.g., in vitro or in vivo, etc. Where desired, vectors, such as viral vectors, may be employed to engineer the cell to express the chimeric proteins as desired. Protocols of interest include those described in published PCT application WO1999/041258, the disclosure of which protocols are herein incorporated by reference.

Depending on the nature of the target cell and/or expression construct, protocols of interest may include electroporation, particle gun technology, calcium phosphate precipitation, direct microinjection, viral infection and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995. In some embodiments, lipofectamine and calcium mediated gene transfer technologies are used. After the subject nucleic acids have been introduced into a cell, the cell is may be incubated, normally at 37° C., sometimes under selection, for a period of about 1-24 hours in order to allow for the expression of the chimeric protein. In mammalian target cells, a number of viral-based expression systems may be utilized to express a subject chimeric proteins. In cases where an adenovirus is used as an expression vector, the chimeric protein coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the chimeric protein in infected hosts. (e.g., see Logan & Shenk, Proc. Natl. Acad. Sci. USA 81:355-359 (1984)). The efficiency of expression may be enhanced by the inclusion of appropriate transcription enhancer elements, transcription terminators, etc. (see Bittner et al., Methods in Enzymol. 153:51-544 (1987)).

Where long-term, high-yield production of the chimeric proteins is desired stable expression protocols may be used. For example, cell lines, which stably express the chimeric protein, may be engineered. Rather than using expression vectors which contain viral origins of replication, host cells can be transformed with chimeric protein expression cassettes and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into a chromosome and grow to form foci which in turn can be cloned and expanded into cell lines. Such engineered cell lines may be particularly useful in screening and evaluation of compounds that interact directly or indirectly with the antibody molecule. In addition, the ASC responsive inducers can be inserted by means of zinc finger nucleases or homologous recombination into “safe harbor” regions of the human or other genomes. Safe harbor regions of interest include regions that are single copy and are not near genes that regulate growth or are likely to cause cancerous transformation or other non-therapeutic perturbations if not properly regulated.

As desired, target cells may be engineered in vitro or in vivo. For target cells that are engineered in vitro, such cells may ultimately be introduced into a host organism. Depending upon the nature of the cells, the cells may be introduced into a host organism, e.g. a mammal, in a wide variety of ways. Hematopoietic cells may be administered by injection into the vascular system, there being 10⁴ or more cells and in some instances 10^{I0} or fewer cells, such as 10⁸ or fewer cells. The number of cells which are employed will depend upon a number of circumstances, the purpose for the introduction, the lifetime of the cells, the protocol to be used, for example, the number of administrations, the ability of the cells to multiply, the stability of the therapeutic agent, the physiologic need for the therapeutic agent, and the like. Alternatively, with skin cells which may be used as a graft, the number of cells would depend upon the size of the layer to be applied to the burn or other lesion. Generally, for myoblasts or fibroblasts, the number of cells will at least about 10⁴ and not more than about 10⁸ and may be applied as a dispersion, generally being injected at or near the site of interest. The cells will usually be in a physiologically-acceptable medium.

In some instances, the target cells are iPS cells. In these instances, fibroblasts or other cell types collected from a patient are harvested and then converted to iPS cells. Any convenient protocol may be employed for converting the harvested cells to iPS cells, where protocols of interest include, but are not limited to, those described in: in United States Patent Application Publication Nos. 20090047263; 20090081784; 20090068742; 20090227032; 20090191159; 20080280362; 20080233610, as well as PCT Application Publication Nos. WO08124133 and WO07069666. Following iPS cell production, the iPS cells then have the activator and target genes inserted at separate loci by homologous recombination. The sites for homologous recombination are those that do not induce onogenic transformation or compromise the ability of the specific cell type to function. These transformed iPS cells are then induced to differentiate into the therapeutic cell type of interest, following which the cells are administered to the patient. This route of therapy has the advantage of avoiding immune rejection. In some embodiments, the ASC-inducible constructs are inserted into fibroblasts that are then directly differentiated into neurons, muscle cells or other cell types using techniques reported in the literature.

### ASC-Induced Proximity

Aspects of the invention include contacting a target cell, e.g., as described above, with an ASC inducer compound in a manner sufficient to induce proximity of at least a first and second chimeric compound, e.g., as described above. Any convenient protocol for contacting the ASC inducer compound with the target cell may be employed. The particular protocol that is employed may vary, e.g., depending on whether the target cell is in vitro or in vivo.

For in vitro protocols, contact of the ASC inducer compound with the target cell may be achieved using any convenient protocol. For examples, target cells may be maintained in a suitable culture medium, and the ASC inducer compound introduced into the culture medium.

For in vivo protocols, any convenient administration protocol may be employed. Depending upon the binding affinity of the ASC inducer compound, the response desired, the manner of administration, the half-life, the number of cells present, various protocols may be employed. The ASC inducer compound may be administered parenterally or orally. The number of administrations will depend upon the factors described above. The ASC inducer compound may be taken orally as a pill, powder, or dispersion; bucally; sublingually; injected intravascularly, intraperitoneally, subcutaneously; by inhalation, or the like. The precise dose and particular method of administration will vary and may be readily determined by the attending physician or human or animal healthcare provider, e.g., the dose and method may be determined empirically. The particular dosage of the ASC inducer compound for any application may be determined in accordance with the procedures used for therapeutic dosage monitoring, where maintenance of a particular level of expression is desired over an extended period of times, for example, greater than about two weeks, or where there is repetitive therapy, with individual or repeated doses of ASC inducer compound over short periods of time, with extended intervals, for example, two weeks or more. A dose of the ASC inducer compound within a predetermined range would be given and monitored for response, so as to obtain a time-expression level relationship, as well as observing therapeutic response. Depending on the levels observed during the time period and the therapeutic response, one could provide a larger or smaller dose the next time, following the response. This process would be iteratively repeated until one obtained a dosage within the therapeutic range. Where the ASC inducer compound is chronically administered, once the maintenance dosage of the ASC inducer compound is determined, one could then do assays at extended intervals to be assured that the cellular system is providing the appropriate response and level of the expression product.

In those instances where the expression of the chimeric molecules is inducible, the methods may further include contacting the cell with a suitable inducer. Accordingly, in some methods, the ASC inducer compound and the inducer of chimeric molecule expression are both contacted with the cell, either sequentially or simultaneously as desired. Such embodiments may provide for enhanced control, e.g., when the ASC inducer may be contacted with a target cell as part of an host organisms diet, etc.

In some embodiments, target cells comprising first and second chimeric molecules, e.g., as described above, are employed to detect the presence of an ASC inducer in a sample. For example, a cell that expresses first and second chimeric proteins each comprising an ASC inducer domain and an effector domain, e.g., as described above, may be provided. The cells further include a reporter construct whose transcription is activated upon proximity induction of the effector domains of the chimeric molecules. In such embodiments, the cells are contacted with the sample and expression of the reporter construct is evaluated to determine whether the ASC inducer compound is present in the sample. The detection may be in the form of a qualitative or quantitative result. For example, where cells are transfected stably with the ABA and PYL chimeric proteins on one construct and a suitable indicator gene (such as luciferase whose expression is response to DNA binding and transcription activator domains of the chimeric proteins), on another construct may be exposed to serum from patients given ABA for a therapeutic purpose, e.g., to cause expression of a therapeutic gene that is responsive to induced proximity of chimeric proteins. The degree of production of the indicator gene, such as luciferase, is then used as an indication of the serum level of ABA.

In some embodiments, the above ASC inducer detection methods, e.g., as described above, are employed in conjunction with research or therapeutic applications to determine levels of ASC inducer in a sample from a subject, e.g., serum from a laboratory animal or a patient, and obtain a measure of the desired result of induced proximity, e.g., expression of a target gene, such as a therapeutic gene whose expression is under the control of DNA binding and activator domains of induced chimeric molecules. In the laboratory setting, such methods may find use in evaluating expression of the therapeutic gene of interest in transgenic non-human animals. In therapeutic applications, ASC inducer screening methods, e.g., as described above, find use in monitoring a treatment protocol, e.g., in providing a measure of proximity induced chimeric molecule activation of therapeutic gene expression.

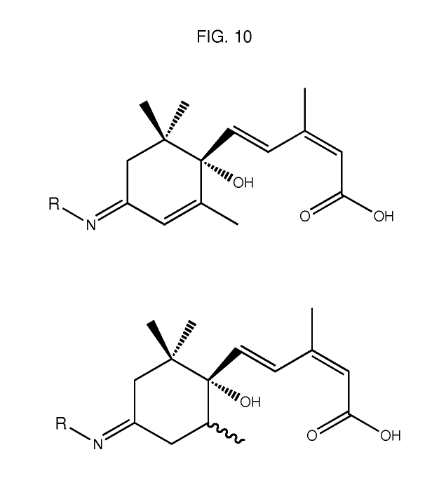

In the event that the induced activity, e.g., transcriptional activation, of the ASC inducer is to be reversed or terminated quickly, an antagonist which can compete with the ASC inducer may be administered. Any convenient antagonist may be employed. Examples of antagonists of interest include, but are not limited to:

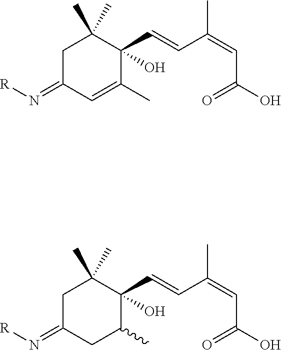

where R is independently selected from an alkyl, an aryl, an alkenyl, an alkynyl, a carbonyl, an acyl, a halogen, a hydroxy, an alkoxy, an aryloxy, a heterocyclic group. See also FIG. 10.

Thus, in the case of an adverse reaction or the desire to terminate the therapeutic effect, an antagonist to the ASC inducer can be administered in any convenient way. Alternatively, one may provide for the presence of an inactivation domain (or transcriptional silencer) with an ASC inducer compound binding domain. In another approach, cells may be eliminated through apoptosis via signalling through Fas or TNF receptor as described elsewhere. See International Patent Applications PCT/US94/01617 and PCT/US94/08008.

### Utility

ASC-induced proximity of chimeric molecules finds use in a variety of different applications. Applications in which ASC induced proximity of chimeric molecules find use include, but are not limited to: regulatable activation or repression of transcription of a desired gene, deletion of a target gene, actuating apoptosis, or triggering other biological events in engineered cells growing in culture or in whole organisms, including in gene therapy applications. The following sections provide additional non-limiting examples of certain applications in which ASC-induced proximity finds use.

In some embodiments, ASC-induced proximity is employed to achieve regulated expression of a target gene, e.g., a therapeutic target gene, for example in the context of gene therapy. The target gene may be a naturally occurring gene or a recombinant gene, as desired. In such embodiments, first and second chimeric proteins and an ASC inducer compound may be employed to activate transcription of an expression construct responsive to a DNA binding domain and transcription activation domain of the first and second chimeric proteins. In these embodiments, the first chimeric protein will include an ASC inducer specific binding domain and a DNA binding domain. The second chimeric protein will include an ASC inducer specific binding domain and a transcription activation domain. In these applications, nucleic acid molecules encoding and capable of directing the expression of these chimeric proteins are introduced into cells in which expression is desired. Also introduced into the cells is construct encoding a target gene linked to a DNA sequence to which the DNA-binding domain of the chimeric proteins is capable of binding. Contacting the engineered cells or their progeny with the ASC-inducer (e.g., by administering it to the animal or patient) leads to assembly of the transcription factor complex and hence to expression of the target gene. In some instances, the level of target gene expression is a function of the number or concentration of chimeric transcription factor complexes, which may in turn be a function of the concentration of the ASC inducer compound, where in some instances ASC inducer compound dose-responsive gene expression is achieved.

Expression of a variety of different target genes may be effected in such embodiments. Examples of target genes of interest include, but are not limited to: factor VIII, factor IX, β-globin, low-density lipoprotein receptor, adenosine deaminase, purine nucleoside phosphorylase, sphingomyelinase, glucocerebrosidase, cystic fibrosis transmembrane conductance regulator, α1-antitrypsin, CD-18, ornithine transcarbamylase, argininosuccinate synthetase, phenylalanine hydroxylase, branched-chain α-ketoacid dehydrogenase, fumarylacetoacetate hydrolase, glucose 6-phosphatase, α-L-fucosidase, β-glucuronidase, α-L-iduronidase, galactose 1-phosphate uridyltransferase, interleukins, cytokines, small peptides etc. In some instances, methods and compositions of the invention are employed to induce production of TNF inhibitors in immune compatible cells, where examples of such TNF inhibitors include, but are not limited to: etanercept, infliximab, and adalimumab.

In yet other embodiments, endogenous genes can be silenced or activated with the ASC-inducer using engineered DNA binding domains (based on zinc fingers) that bind near a gene of interest or that bind to a pathogenic virus such as HIV. Administering the ASC-inducer in these embodiments recruits either a transcriptional activator or a transcriptional repressor leading to desired modulation of the activity of the endogenous gene.

ASC inducer mediated proximity also finds use in selective activation or inactivation of signaling pathways. For example, ASC inducer proximity in accordance with the invention may be employed to activate of a variety of different signaling pathways, e.g., by localizing a factor of the pathway to particular cellular location (such as SOS to the membrane for ras pathway activation, localizing NF-KB or NFAT to the nucleus to enhance immune responses, etc.). Conversely, ASC inducer proximity in accordance with the invention may be employed to inactivate a variety of different signaling pathways, e.g., by localizing a factor of the pathway to particular cellular location (such localizing NF-KB out of the nucleus to inhibit inflammation, etc.).

ASC inducer mediated proximity also finds use in the production of recombinant proteins and viruses. As such, aspects of the invention include use of ASC inducer mediated proximity for the production of recombinant therapeutic proteins, e.g., for commercial and investigational purposes. Examples of proteins of interest include, but are not limited to: erythropoietin, tissue plasminogen activator, clotting factors such as Factor VIII or anti-coagulant factors such as Protein C; anti-angiogenic molecules for treatment of cancer, including fragments of the VEGF receptor, growth factors, cell fate inducers, morphogens such as Hedgehog or antibodies, etc. Embodiments of the invention may be used to tightly control expression of such proteins in host cell to achieve expression at specific desired times and/or levels. Similarly, embodiments of the invention may be employed in viral production, e.g., to achieve expression at desired times and/or levels of viral proteins in packaging cells, etc., e.g., to address host cell toxicity resulting from expression of such proteins.

ASC inducer mediated proximity in accordance with aspects of the invention also finds use in a wide range of biological experiments in which precise control over expression of a target gene is desired. Such applications include, but are not limited to: (1) expression of a protein or RNA of interest for biochemical purification; (2) regulated expression of a protein or RNA of interest in tissue culture cells (or in vivo, via engineered cells) for the purposes of evaluating its biological function; (3) regulated expression of a protein or RNA of interest in transgenic animals for the purposes of evaluating its biological function; (4) regulating the expression of a gene encoding another regulatory protein, ribozyme or antisense molecule that acts on an endogenous gene for the purposes of evaluating the biological function of that gene; and (5) regulating biomolecule activities or cellular processes by controlling localization of biomolecules. Transgenic animal models and other applications in which the components of this invention may be adapted include those disclosed in PCT/US95/10591.

Further details regarding applications various application in which ASC-induced multimerization in accordance with embodiments of the invention may be employed included, but are not limited to, those described in WO94/18317, WO95/02684, WO96/20951, WO95/41865, and WO 99/041258.

### Pharmaceutical Compositions

Also provided are pharmaceutical preparations. Pharmaceutical preparations are compositions that include an ASC inducer compound (for example abscisic acid) present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a mammal, the compounds and compositions of the invention and pharmaceutically acceptable vehicles, excipients, or diluents may be sterile. In some instances, an aqueous medium is employed as a vehicle when the compound of the invention is administered intravenously, such as water, saline solutions, and aqueous dextrose and glycerol solutions.

Pharmaceutical compositions can take the form of capsules, tablets, pills, pellets, lozenges, powders, granules, syrups, elixirs, solutions, suspensions, emulsions, suppositories, or sustained-release formulations thereof, or any other form suitable for administration to a mammal. In some instances, the pharmaceutical compositions are formulated for administration in accordance with routine procedures as a pharmaceutical composition adapted for oral or intravenous administration to humans. Examples of suitable pharmaceutical vehicles and methods for formulation thereof are described in Remington: The Science and Practice of Pharmacy, Alfonso R. Gennaro ed., Mack Publishing Co. Easton, Pa., 19th ed., 1995, Chapters 86, 87, 88, 91, and 92, incorporated herein by reference.

The choice of excipient will be determined in part by the particular compound, as well as by the particular method used to administer the composition. Accordingly, there is a wide variety of suitable formulations of the pharmaceutical composition of the present invention.

Administration of the ASC inducer compounds of the invention may be systemic or local. In certain embodiments administration to a mammal will result in systemic release of a compound of the invention (for example, into the bloodstream). Methods of administration may include enteral routes, such as oral, buccal, sublingual, and rectal; topical administration, such as transdermal and intradermal; and parenteral administration. Suitable parenteral routes include injection via a hypodermic needle or catheter, for example, intravenous, intramuscular, subcutaneous, intradermal, intraperitoneal, intraarterial, intraventricular, intrathecal, and intracameral injection and non-injection routes, such as intravaginal rectal, or nasal administration. In particular embodiments, the compounds and compositions of the invention are administered orally. In particular embodiments, it may be desirable to administer one or more compounds of the invention locally to the area in need of treatment. This may be achieved, for example, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

The ASC inducer compounds can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

In some embodiments, formulations suitable for oral administration can include (a) liquid solutions, such as an effective amount of the compound dissolved in diluents, such as water, or saline; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are described herein.

The subject formulations of the present invention can be made into aerosol formulations to be administered via inhalation. These aerosol formulations can be placed into pressurized acceptable propellants, such as dichlorodifluoromethane, propane, nitrogen, and the like. They may also be formulated as pharmaceuticals for non-pressured preparations such as for use in a nebulizer or an atomizer.

In some embodiments, formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration may be presented as creams, gels, pastes, or foams, containing, in addition to the active ingredient, such carriers as are appropriate. In some embodiments the topical formulation contains one or more components selected from a structuring agent, a thickener or gelling agent, and an emollient or lubricant. Frequently employed structuring agents include long chain alcohols, such as stearyl alcohol, and glyceryl ethers or esters and oligo(ethylene oxide) ethers or esters thereof. Thickeners and gelling agents include, for example, polymers of acrylic or methacrylic acid and esters thereof, polyacrylamides, and naturally occurring thickeners such as agar, carrageenan, gelatin, and guar gum. Examples of emollients include triglyceride esters, fatty acid esters and amides, waxes such as beeswax, spermaceti, or carnauba wax, phospholipids such as lecithin, and sterols and fatty acid esters thereof. The topical formulations may further include other components, e.g., astringents, fragrances, pigments, skin penetration enhancing agents, sunscreens (i.e., sunblocking agents), etc.

A compound of the invention may be formulated for topical administration. The vehicle for topical application may be in one of various forms, e.g. a lotion, cream, gel, ointment, stick, spray, or paste. They may contain various types of carriers, including, but not limited to, solutions, aerosols, emulsions, gels, and liposomes. The carrier may be formulated, for example, as an emulsion, having an oil-in-water or water-in-oil base. Suitable hydrophobic (oily) components employed in emulsions include, for example, vegetable oils, animal fats and oils, synthetic hydrocarbons, and esters and alcohols thereof, including polyesters, as well as organopolysiloxane oils. Such emulsions also include an emulsifier and/or surfactant, e.g. a nonionic surfactant to disperse and suspend the discontinuous phase within the continuous phase.

Suppository formulations are also provided by mixing with a variety of bases such as emulsifying bases or water-soluble bases. Formulations suitable for vaginal administration may be presented as pessaries, tampons, creams, gels, pastes, foams.

A compound of the invention may also be formulated as a dietary supplement or nutraceutical, e.g., for oral administration. For a nutraceutical formulation, or an oral pharmaceutical formulation, suitable excipients include pharmaceutical grades of carriers such as mannitol, lactose, glucose, sucrose, starch, cellulose, gelatin, magnesium stearate, sodium saccharine, and/or magnesium carbonate. For use in oral liquid formulations, the composition may be prepared as a solution, suspension, emulsion, or syrup, being supplied either in solid or liquid form suitable for hydration in an aqueous carrier, such as, for example, aqueous saline, aqueous dextrose, glycerol, or ethanol, preferably water or normal saline. If desired, the composition may also contain minor amounts of non-toxic auxiliary substances such as wetting agents, emulsifying agents, or buffers. A compound of the invention may also be incorporated into existing nutraceutical formulations, such as are available conventionally, which may also include an herbal extract.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may include the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Dose levels can vary as a function of the specific compound, the nature of the delivery vehicle, and the like. Desired dosages for a given compound are readily determinable by a variety of means.

The dose administered to an animal, particularly a human, in the context of the present invention should be sufficient to effect a prophylactic or therapeutic response in the animal over a reasonable time frame, e.g., as described in greater detail below. Dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound.

In pharmaceutical dosage forms, the ASC inducer compounds may be administered in the form of a free base, their pharmaceutically acceptable salts, or they may also be used alone or in appropriate association, as well as in combination, with other pharmaceutically active compounds.

In those instances where the ASC inducer is ABA, the ASC inducer may be present in a given dosage form in an amount that is greater than the amount of ABA found in an plant extract, such castor oil. In some instances where the ASC inducer is ABA, the pharmaceutical composition is not a plant extract, such as castor oil or does not include a plant extract, such as castor oil.

### Kits

Also provided are kits for use in practicing the subject methods, where the kits may include one or more of the above components, e.g., nucleic acids encoding chimeric proteins, vectors including the same, ASC inducer compounds, host cells, cells for use in ASC inducer detection, and the like. The various kit compounds may be present in separate containers in the kits, or combined as desired.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the Internet to access the information at a removed site. Any convenient means may be present in the kits.

The following examples are offered by way of illustration and not by way of limitation.

### EXPERIMENTAL

### I. Materials and Methods

Vectors for Transcriptional Activation.

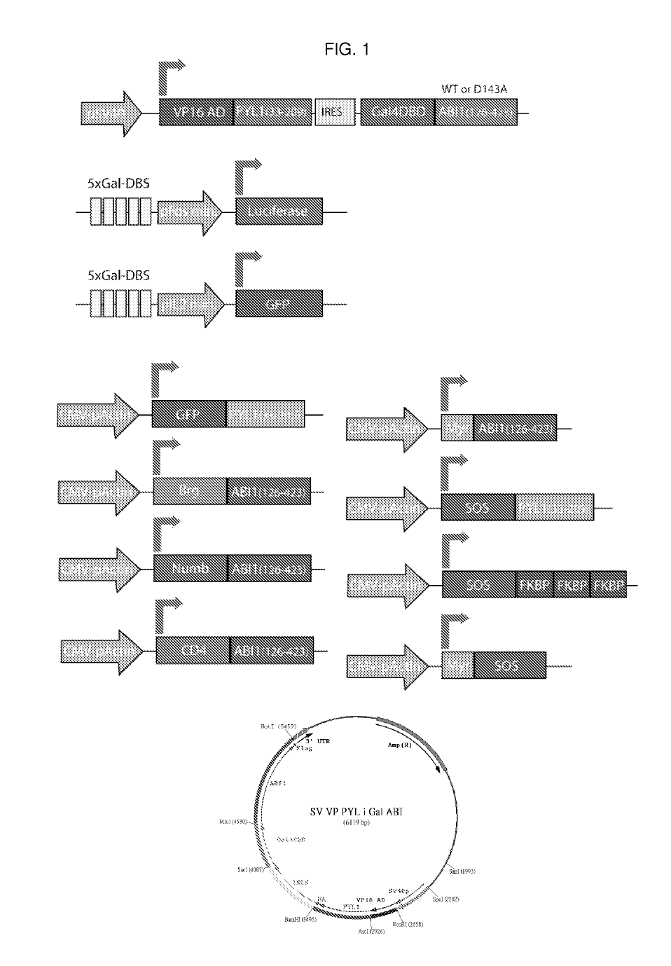

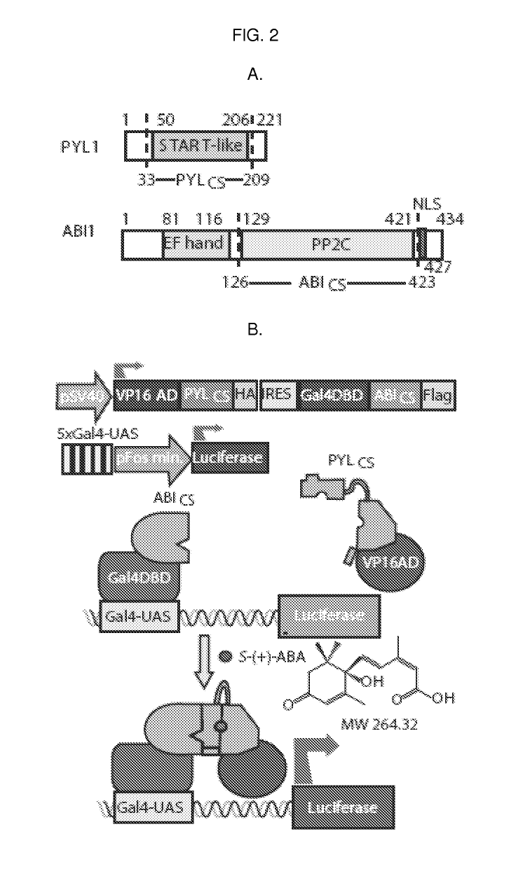

Activation construct: A fragment of PYL1 (one of PYR/PYL/RCAR members) (aa 33-209, also referred to as “PYLcs”) was linked to a transcriptional activation domain and a fragment of ABI1 (one of PP2C members) (aa 126-423, SEQ ID NO:2, also referred to as “ABIcs”) was linked to a DNA binding domain using recombinant DNA methods. A map of the resultant DNA construct is shown in FIG. 1 and FIG. 2. In the constructs illustrated in FIG. 1 and FIG. 2, PYL1 (“PYLcs”) is fused to the VP16 activation domain and ABI1 (“ABIcs”) is fused to the Gal4 DNA binding domain. These two regulatory elements are linked by IRES and driven by the SV40 early promoter. Reporter constructs: Full length Luciferase is driven by Fos gene minimal promoter with 5 copies of Gal4 DNA binding sites immediately upstream of Fos promoter. GFP reporter is driven by IL2 minimal promoter with 5×Gal4 DNA binding sites upstream.

Vectors for Cellular Protein Re-Localization and MAP Kinase Activation.

All of these constructs, as shown in FIG. 1, are driven by CMV-enhancer and chicken beta-actin promoter. A fragment of ABI1 (aa 126-423) is either fused to full length mouse Brg, full length mouse Numb, full length human CD4 or myristoylation signal. A fragment of PYL1 (aa 33-209) is fused to full length GFP or SOS. Full length SOS is fused either to myristoylation signal or three copies of FKBP12.

Plasmid Construction.

All DNA fragments were amplified by polymerase chain reaction (PCR) from other intermediate constructs or genomic DNA or complementary DNA (cDNA) purchased from the Arabidopsis Biological Resource Center at Ohio State University or Open BioSystem. PCR was performed with Phusion DNA Polymerase (New England Biolabs), Expand Long Template PCR System (Roche), or In-Fusion PCR Cloning Kit (Clontech) with MJ Research Peltier Thermal Cycler (Bio-Rad). All constructs were made by inserting into or replacing parts of the actin-IRES (internal ribosomal entry site)-eGFP (enhanced GFP) vector (J. I. Wu, et al. (2007) Neuron 56, 94-108), myr-FKBP (MF3E) vector (Spencer, D. M. et al. (1995) Proc. Natl. Acad. Sci. U.S.A. 92, 9805-9809), or pGEX vector (GE Healthcare) with the amplified DNA fragments. All the plasmids were sequenced to confirm the sequence and avoid cloning errors. Primers used for preparing constructs used in this study were as follows:

ABI forward (F):
(SEQ ID NO: 13)
5′-CCGACAACGCGTGTGCCTTTGTATGGTTTTACTTC-3′;
ABI FLAG reverse (R):
(SEQ ID NO: 14)
5′-CCGACAGCGGCCGCTCACTTATCGTCATCGTCCTTGT
AATCCTTCAAATCAACCACCACCACAC-3′;
GFP K F:
(SEQ ID NO: 15)
5′-CCGACAGTCGACGCCACCATGGTGAGCAAGGGCGAGGAG-3′;
GFP R:
(SEQ ID NO: 16)
5′-CCGACAGGCGCGCCCTTGTACAGCTCGTCCATGCC-3′;
PYL F:
(SEQ ID NO: 17)
5′-CCGACAGGCGCGCCAACTCAAGACGAATTCACCCAAC-3′;
PYL HA R:
(SEQ ID NO: 18)
5′-CCGACAGCGGCCGCTCAAGCGTAATCTGGAACATCGTATGGG
TAGTTCATAGCTTCAGTGATCGAAG-3′;
m-Numb F:
(SEQ ID NO: 19)
5′-CCGACAGTCGACATGAACAAACTACGGCAAAGCTTC-3′;
m-Numb R:
(SEQ ID NO: 20)
5′-CCGACAACGCGTACCCCCACCAGAACCCCCACCAGAAAG
TTCTATTTCAAACGTTTTC-3′;
hCD4 F:
(SEQ ID NO: 21)
5′-CCGACAACGCGTAATGGGGCTACATGTCTTCTGA-3′;
hCD4 R:
(SEQ ID NO: 22)
5′-CCGACAGCGGCCGCAATGGGGCTACATGTCTTCTGA-3′;
SOS F:
(SEQ ID NO: 23)
5′-CCGACAGTCGACGCCACCATGCAGGCGCAGCAGCTGCCCTAC-3′;
SOS R:
(SEQ ID NO: 24)
5′-CCGACAGGCGCGCCGGAAGAATGGGCATTCTCCAAC-3′;
ABIc NLS R:
(SEQ ID NO: 25)
5′-CCGACAGCGGCCGCTCATACCTTTCTCTTCTTTTTTGGATCTA
CCTTTCTCTTCTTTTTTGGATCGTTCAAGGGTTTGCTCTTGAG-3′;
ABI1 myr F:
(SEQ ID NO: 26)
5′-CCGACAGAATTCGCCACCATGGGTAGCAACAAGAGCAAGGG
AGGTGTGCCTTTGTATGGTTTTACTTC-3′;
Frb F:
(SEQ ID NO: 27)
5′-CCGACAGGCGCGCCTGGAATGTGGCATGAAGGCCTGGAA-3′;
Frb R:
(SEQ ID NO: 28)
5′-CCGACAGCGGCCGCTCACTGCTTTGAGATTCGTCGGAACAC-3′;
VP16 F:
(SEQ ID NO: 29)
5′-CCGACAGAATTCGCCACCATGGGCCCTAAAAAGAAGCGTAAAG-3′;
VP16 R:
(SEQ ID NO: 30)
5′-CCGACAGGCGCGCCTCCCACCGTACTCGTCAATTCCAAG-3′;
Ga14 F:
(SEQ ID NO: 31)
5′-CCGACAGAGCTCATGAAGCTACTGTCTTCTATCG-3′;
Ga14 R:
(SEQ ID NO: 32)
5′-CCGACAACGCGTCGATACAGTCAACTGTCTTTGAC-3′;
ABI D143A F:
(SEQ ID NO: 33)
5′-CCGACAACGCGTATGGTGCCTTTGTATGGTTTTACTTCGATT
TGTGGAAGAAGACCTGAGATGGAAGCTGCTGTTTCGACTATAC-3′;
ABI1 G F:
(SEQ ID NO: 34)
5′-CCGACAGGATCCGTGCCTTTGTATGGTTTTACTTC-3′;
ABI1 G R:
(SEQ ID NO: 35)
5′-CCGACAGGATCCTCACTTCAAATCAACCACCACCACAC-3′;
simian virus 40 (SV40) F:
(SEQ ID NO: 36)
5′-CCGACAACTAGTCTGTGGAATGTGTGTCAGTTAG-3′;
SV40 R:
(SEQ ID NO: 37)
5′-CCGACAGAATTCCGAAAATGGATATACAAGCTCC-3′;
hemagglutinin (HA) R:
(SEQ ID NO: 38)
5′-CCGACAGGATCCTCAAGCGTAATCTGGAACAT
CGTATG-3′.

Cell Culture and Transfection.

CHO, HEK 293T, NIH 3T3, B35, MEF, and COS7 cells were cultured in Dulbecco's modified Eagle's medium (DMEM) (Gibco) with 10% FBS (Omega Scientific), 1× glutamate (Gibco), and 1× penicillin/streptomycin (Pen/Strep; Gibco). Jurkat cells were cultured in RPMI 1640 (Gibco) with 10% FBS, 1× glutamate, and 1×Pen/Strep. E14 and TC1 were cultured in Knockout DMEM (Gibco) with 7.5% ES FBS (Applied Stem Cell), 7.5% knockout SR (Gibco), 1× Hepes (Gibco), 1× sodium pyruvate (Gibco), 1×MEM NEAA (nonessential amino acids; Gibco), 1×2-mercaptoethanol (Gibco), 1× glutamate, 1×Pen/Strep, and leukemia inhibitory factor (LIF) on gelatin (Millipore)-coated plates. Cells (50,000 to 200,000) were plated in a well of a 24-well plate the day before transfection. An amount of 0.1 to 0.5 μg of DNA of each construct was mixed with 50× (v/w) Opti-MEM (Gibco), and then 3× (v/w) FuGENE HD (Roche) was added and thoroughly mixed. After incubation at room temperature for 20 min, the mixture was added to the cells and cultured for 24 to 48 hours before experiments were performed.

Luciferase Assay.

Cells in 24-well plates were lysed with 100 μl of 1× Passive Lysis Buffer (Promega) at room temperature for 10 min on a shaker (˜80 rpm). Cell lysates were then spun down (16,000 g) and 10 μl of supernatant was used for luciferase assay. Luciferase substrate solution [100 μl; 5 mg of D-luciferin (BD Biosciences) and 7 mg of coenzyme A (Sigma) in 33 ml of luciferase reading buffer: 20 mM tricine, 1.07 mM (MgCO₃)₄Mg(OH)2.5H2O, 2.67 mM MgSO₄, 0.1 mM EDTA, 33.3 mM dithiothreitol (DTT), and 0.53 mM ATP in water] was added to the cell lysates, and signal was read with a 3-s delay and 1-s integration with a Turner BioSystem Modulus microplate reader. Obtained data were analyzed by Prism 5 (GraphPad Software).

Immunofluorescence Staining.

Cells grown on glass coverslips were washed in phosphate-buffered saline (PBS) and fixed with 4% paraformaldehyde (PFA, prepared in PBS) at room temperature for 20 min. After three 5-min washes in PBS, the cells were permeabilized with 5% normal goat serum and 0.1% Triton X-100 in PBS at room temperature for 30 min and incubated with primary antibodies (FLAG antibody M2, Agilent, cat. #200472-21) at 4° C. overnight in the same buffer. The slides were subsequently washed three times with PBS (5 min each wash) and then incubated with Alexa fluorophore 594-conjugated secondary antibodies (Molecular Probes) for 1 hour at room temperature in the dark. Slides were washed three times in PBS for 5 min. Finally, the slides were mounted in Vectashield containing 4′,6-diamidino-2-phenylindole (DAPI; H-1500, Vector Laboratories) and imaged (63×) with a Leica DM5000B microscope.

Fluorescence Quantification.

The area and integrated fluorescence intensity of the whole cell and the nucleus were measured with Fiji (an open-source image processing package based on ImageJ) for images from the cytoplasmic or nuclear localization experiments. The area and the integrated fluorescence intensity of the cytoplasm were calculated by subtracting the value of the nucleus from the whole cell. The mean fluorescence intensity of nucleus or cytoplasm was calculated by dividing the integrated fluorescence intensity with the area of each subcellular compartment. The ratio between nuclear or cytoplasmic distribution was calculated by comparing the corresponding mean fluorescence intensity. Twenty cells were measured in each experiment.

SOS Activation.

Six-well plates of 293T cells containing 0.5×106 cells per well were transfected with 3 μg of total DNA using FuGENE HD (Roche). Cells were transfected with expression plasmids encoding myr-ABI, SOS-PYL, SOS-FKBP, myr-SOS, and an expression vector control, pUC19. ABA (250 μM) was added at 3, 6, 12, and 24 hours before harvesting the cells. Cells were lysed in 1× lysis buffer [50 mM Tris-HCl (pH 7.5), 300 mM NaCl, 1% NP-40, 0.1% SDS, 0.5% sodium deoxycholate, 20 mM NaF, 1 mM Na₃VO₄, 1 mM phenylmethylsulfonyl fluoride (PMSF), and 1× protease inhibitor mixture (Calbiochem)] for 20 min at 4° C. After centrifugation at 14,000 g for 15 min, proteins were separated on 4 to 12% bis-tris NuPAGE gels (Invitrogen) and transferred to polyvinylidene difluoride membranes. After incubation with 10% bovine serum albumin (BSA) in PBST (PBS and 0.1% Tween 20), membranes were incubated with antibody against HA 6E2 (Cell Signaling Technologies, cat. #2367), antibody against Brg H88 (Santa Cruz Biotechnology, cat. #sc-10768), antibody against Hsp90 (BD Biosciences, cat. #610418), antibody that recognizes phosphorylated ERK1/2 (Cell Signaling Technology, cat. #4377), antibody that recognizes total ERK1/2 (Cell Signaling Technology, cat. #9107), and antibody against FLAG M2 (Agilent, cat. #200472-21) in 10% BSA-PBST overnight at 4° C. Infrared dye 800 and 680 anti-rabbit or anti-mouse secondary antibodies were incubated at room temperature for 1 hour in 10% BSA-PBST and detected with a LI-COR/Odyssey scanner.

Phosphatase Activity Assay.

Wild-type and mutant GST-ABIcs fusion proteins were produced from BL21(DE3)pLysS (Promega) and purified by Glutathione Superflow resin (Clontech). Phosphatase activity of GST-ABIcs was measured by Pro Fluor Ser/Thr PPase Assay Kit (Promega, V1260) following the manufacturer's protocol. Briefly, in a 96-well plate, GST-ABIcs was supplied with or without 40 mM MgCl₂ and incubated with fluorophore-conjugated phosphorylated PP2C substrate peptides at room temperature for 30 min. Protease for dephosphorylated peptide was then added and incubated at room temperature for 90 min. Termination buffer was added and the reaction mixture was read at an excitation wavelength of 485 nm and an emission wavelength of 530 nm with a Molecular Device SpectraMax M2 plate reader.

ABA-Dependent Pull-Down.

Wild-type or mutant GST-ABIcs-bound glutathione beads (50 μA were incubated with 400 μg of GFP-PYLcs-containing cell lysates in 200 μl of PBS with or without 500 μM ABA overnight at 4° C. Beads were then washed three times with 1 ml of PBS and then processed for Western blot analysis. GFP-PYLcs and GST-ABIcs were detected with antibodies against GFP (Invitrogen, cat. #A11122) and GST (Upstate, cat. #16-209), respectively.

Mouse Gavage and Serum Collection.

Mice were housed in the Stanford University Research Animal Facility in accordance with federal and institutional guidelines. Mice aged 6 to 8 weeks and weighing about 30 g were orally gavaged with 10 mg of ABA in EtOH/Tween 20/Cremophor (4:3:1; 100 μA or just vehicle with syringes. Blood was collected from the tails of mice at the indicated time after gavage and left on ice for 20 min. After blood was coagulated, the samples were spun down at 1500 g for 20 min and serum was collected for immediate use or snap-frozen and stored at −80° C.

Lymphocyte Proliferation Assay.

Naïve T cells were isolated from spleens of C57BL/6J mice with MACS T cell isolation kit (Miltenyi Biotec). Isolated T cells were stimulated for 72 hours at 37° C. with plate-bound monoclonal antibodies against CD3 (10 μg/ml) and CD28 (2 μg/ml) before cell count measurements.

### II. Inducibility of Gene Activation with the ABA-Induced Proximity System

The inducible gene activation module illustrated in FIG. 1 was introduced into a mammalian cell line (CHO, Chinese Hamster Ovary cells) along with a luciferase reporter to test the inducibility of gene activation by ABA, as shown in FIG. 2.

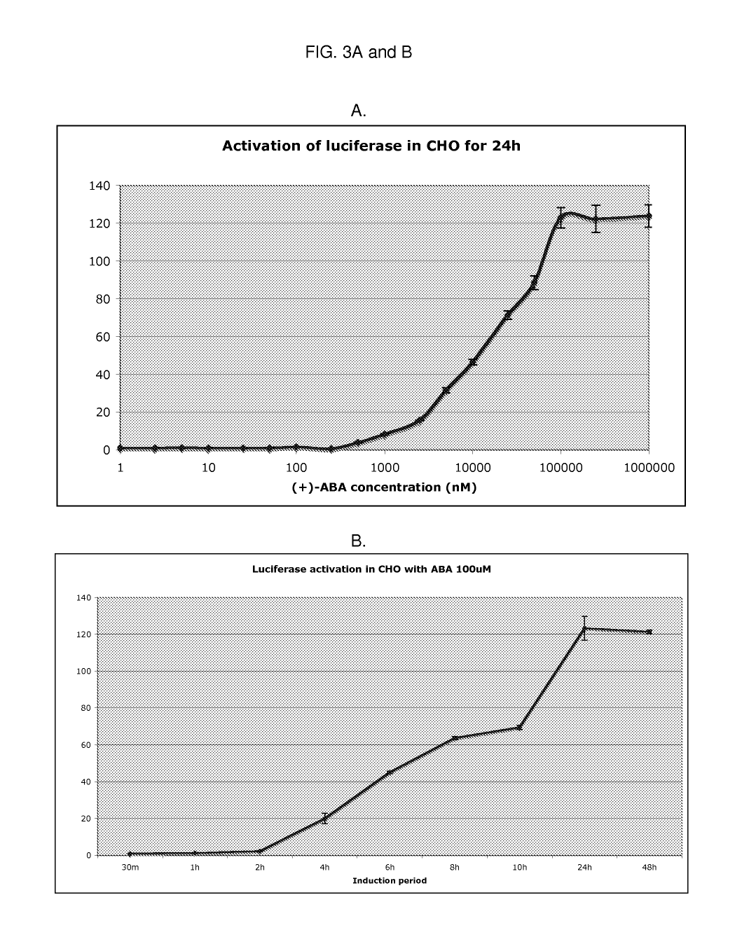

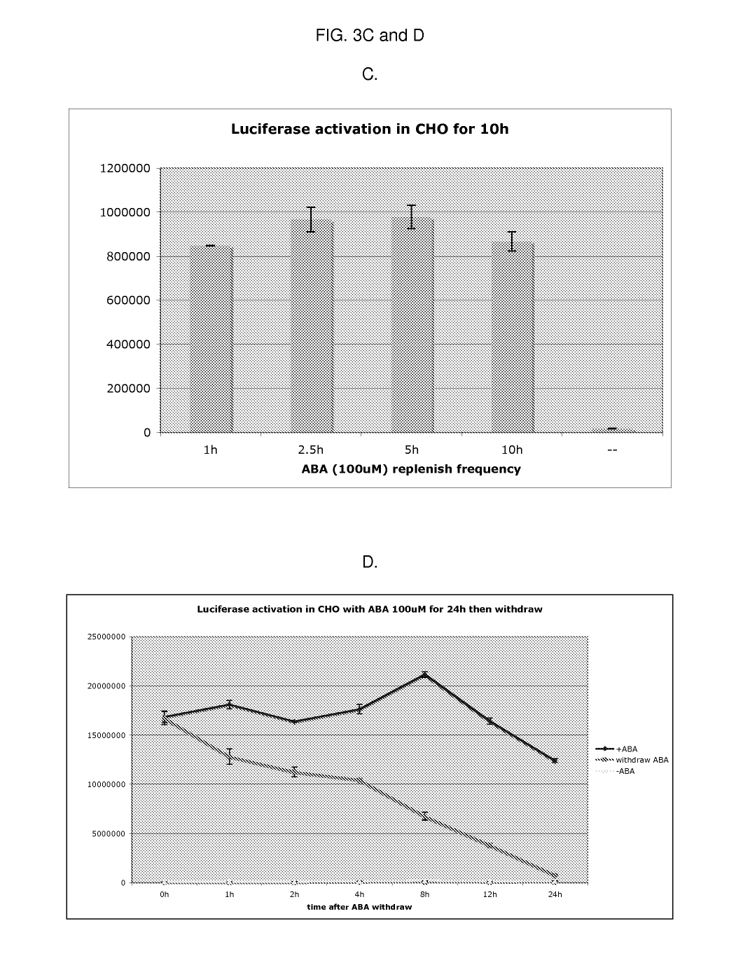

Expression of these molecules in cells is harmless and produces no effects until the addition of ABA, at which point high level transcription of luciferase is rapidly induced within hours (FIGS. 3A, 3B, and 3C) and is fully reversible upon removal of ABA (FIG. 3D). FIG. 3A illustrates the ABA concentration dependence of luciferase activation. Different amounts of ABA were added to the CHO cell culture for 24 hours and cells were assayed for luciferase activity. The cells were pre-transfected with the activation and reporter DNA constructs for 24 hours. Induction fold change is calculated by induced over non-induced luciferase signal. FIG. 3B illustrates time dependence of luciferase activation by ABA. 100 μM of ABA was added to the CHO cell culture and cells were assayed for luciferase activity at indicated time after ABA addition. The cells were pre-transfected with the activation and reporter DNA constructs for 24 hours. FIG. 3D illustrates the results of a reversibility test of ABA-induced luciferase activation. After 100 μM of ABA was added to the CHO cell culture for 24 hours, cells were washed with PBS and cell culture media without ABA and kept growing in ABA-free media for indicated period before assayed for luciferase activity. The cells were pre-transfected with the activation and reporter DNA constructs for 24 hours.

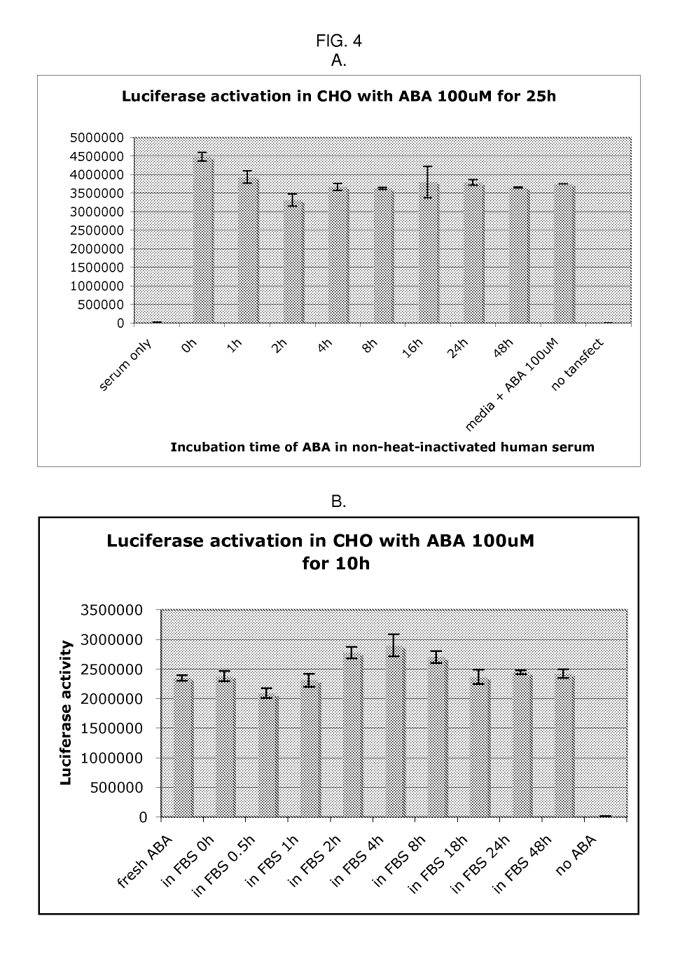

In contrast to inducers of proximity such as C20 methallyl rapamycin, ABA is stable in cell culture media and in pure human or fetal bovine serum for up to 48 hours as there is no significant reduction in the ability of ABA to induce transcription of luciferase, e.g., as illustrated in FIGS. 3C, 4A and 4B. FIG. 3C provides the results of a stability test of ABA in cell culture conditions. CHO cells were pre-transfected with the activation and reporter DNA constructs for 24 hours. 100 μM ABA was then added to the culture and cells were assayed for luciferase activity after 10 hours. During the 10-hour incubation, the cell culture media were replaced with media containing freshly added ABA every 1, 2.5, 5 hour or no replacement. FIG. 4A illustrates the results of an experiment in which human serum pre-incubated ABA was used to induce luciferase transcription. ABA was incubated with pure non-heat inactivated human serum at 37° C. for indicated time period. ABA containing serum was then added to the CHO cell culture to a final 100 μM ABA concentration for 25 hours and cells were assayed for luciferase activity. The cells were pre-transfected with the activation and reporter DNA constructs for 24 hours. FIG. 4B illustrates the results of an experiment in which fetal bovine serum pre-incubated ABA was used to induce luciferase transcription. ABA was incubated with pure heat inactivated fetal bovine serum at 37° C. for indicated time period. ABA containing serum was then added to the CHO cell culture to a final 100 μM ABA concentration for 10 hours and cells were assayed for luciferase activity. The cells were pre-transfected with the activation and reporter DNA constructs for 24 hours.

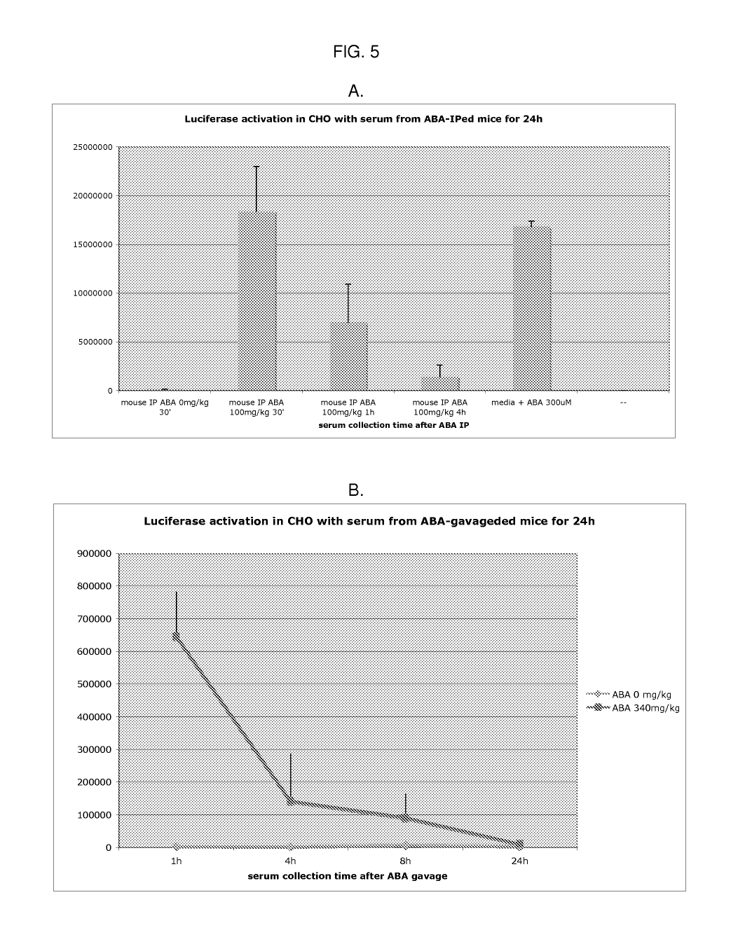

ABA is also bio-available when administered to mice either by intraperitoneal injection or gavage as shown in FIG. 5. FIG. 5A illustrates the bio-availability of ABA in mice by intraperitoneal injection. With 100 mg ABA/kg mouse body weight, ABA is rapidly available in 30 minutes and still remains in the system after 4 hours. ABA was intraperitoneally injected to mice and serum was collected at indicated time. ABA containing serum was then added as 10% (v/v) of culture media to the CHO cell culture for 24 hours and cells were assayed for luciferase activity. The CHO cells were pre-transfected with the activation and reporter DNA constructs for 24 hours before adding serum. FIG. 5B shows the oral bio-availability of ABA in mice by gavage. With 340 mg ABA/kg mouse body weight, ABA is rapidly available in 1 hour, remains in the system after 8 hours and returns to background level after 24 hours. ABA was orally administrated by gavage to mice and serum was collected at indicated time. ABA containing serum was then added as 10% (v/v) of culture media to the CHO cell culture for 24 hours and cells were assayed for luciferase activity. The CHO cells were pre-transfected with the activation and reporter DNA constructs for 24 hours before adding serum.

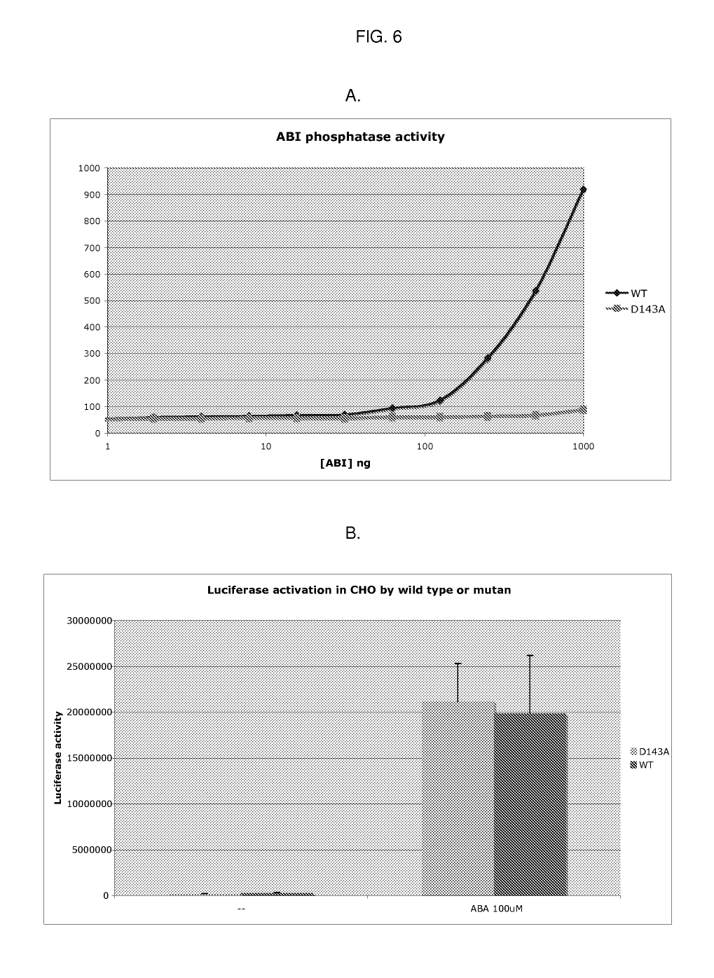

Wild type ABI1 (aa 126-423) contains the phosphatase domain and retains its activity. To eliminate potential toxic effect of the phosphatase activity introduced by expression of ABI1 while retaining its binding ability to PYL1-ABA, a point mutation in ABI1 phosphatase active site, Asp 143 to Ala (D143A) was made to reduce the phosphatase activity and yet retain PYL1-ABA binding. FIG. 6A illustrates that the ABI1 D143A mutant lost phosphatase activity as compared to wild type ABI. GST-ABI wt or GST-ABI D143A fusion proteins were expressed in E. coli. and purified by glutathione beads. A series amount of GST-ABI fusion proteins were then used for phosphatase activity assay following the protocol provided by vendor (Promega). The phosphate activity is positively correlated to fluorescence intensity. FIG. 6B shows that the ABI1 D143A mutant retains its binding to PYL-ABA complex. Mutant ABI (D143A) was comparable to wild type ABI in ABA induced luciferase activation assay. The CHO cells were pre-transfected with the activation constructs with wild type or mutant ABI and luciferase reporter DNA constructs for 24 hours. 100 μM of ABA was then added to the CHO cell culture and cells were assayed for luciferase activity after another 24 h.

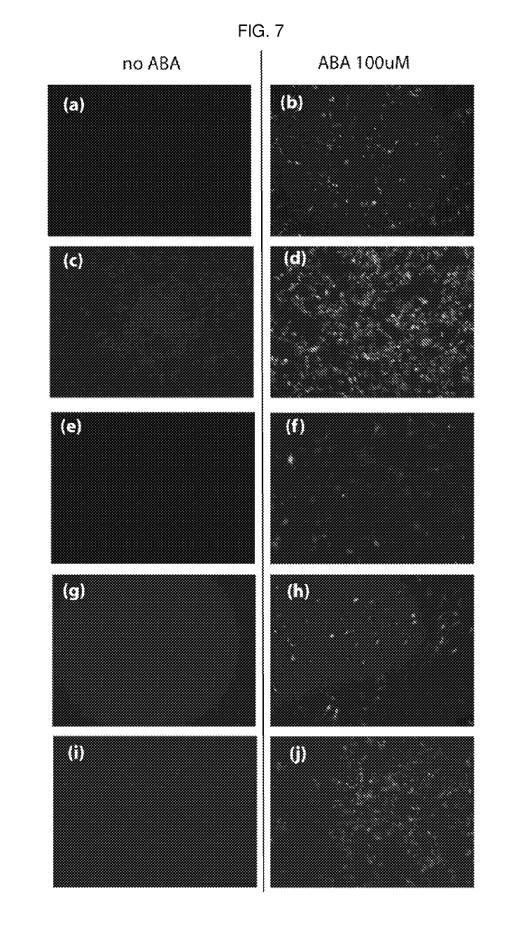

ABA regulated transcriptional activation is applicable to various cell lines from different origins besides CHO cells, as shown in FIG. 7. The GFP reporter only expressed after ABA was added to the cells. In FIG. 7, (a) and (b) CHO cells (Chinese hamster ovary cells); (c) and (d) HEK 293 cells (human embryonic kidney cells); (e) and (f) Cos7 cells (African green monkey kidney cells); (g) and (h) NIH3T3 cells (mouse embryonic fibroblast cells); (i) and (j) TC1 cells (mouse embryonic stem cells). The cells were pre-transfected with the activation construct and GFP reporter construct for 24 hours. 100 μM of ABA was then added to the cell culture and cells were observed under fluorescence microscope for GFP expression after another 24 h.

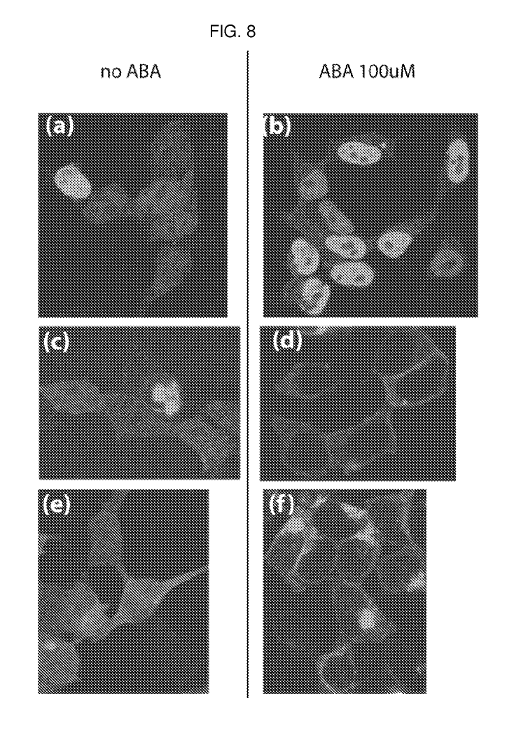

In addition to transcriptional activation, ABA-induced proximity has been successfully applied to control cellular protein localization as illustrated in FIG. 8. In the system of FIG. 8, the GFP reporter is fused to PYL1 (aa 33-209) and ABI1 (aa 126-423) is fused to either Brg (a nuclear protein), Numb (a cytoplasmic protein) or CD4 (a membrane protein). After adding ABA to the cells, GFP was re-localized to specific cellular compartment depending on the ABI fusion proteins. In FIG. 8, (a) and (b) GFP-PYL+Brg-ABI; (c) and (d) GFP-PYL+Numb-ABI; (e) and (f) GFP-PYL+CD4-ABI. The HEK 293 cells were pre-transfected with the localization construct (Brg/Numb/CD4-ABI) and GFP-PYL reporter construct for 24 hours. 100 μM of ABA was then added to the cell culture and cells were observed under confocal microscope for GFP expression after another 24 h.

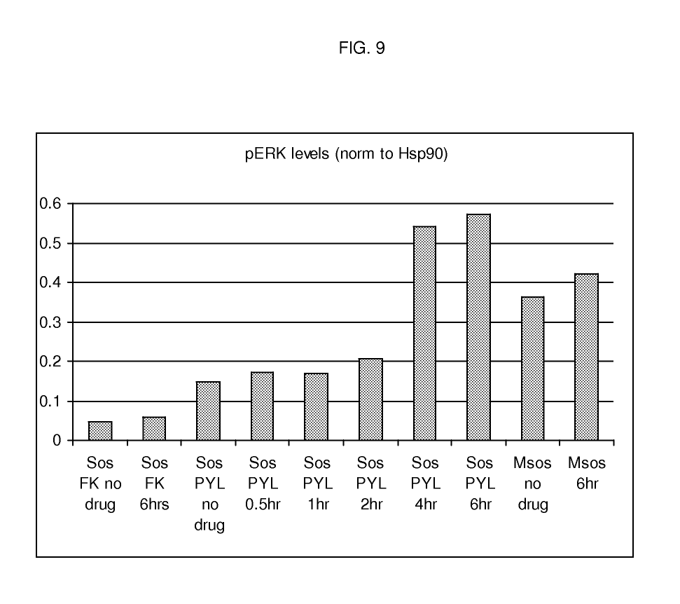

The localization of cellular proteins induced by ABA can regulate biological processes, such as signaling pathways, as demonstrated in FIG. 9. This figure illustrates ABA induced localization of SOS to the cell membrane, which localization induces the MAP kinase signaling pathway. In FIG. 9, HEK 293T cells were co-transfected with myr-SOS (a constitutive active form) only or myr-ABI and either SOS-PYL or SOS-FKBP3 (a negative control). 200 μM ABA was added after 24 hours and cells were collected at the indicated times to probe and quantify the phosphorylated form of Erk protein.

To achieve a quick reverse of ABA induced proximity, ABA antagonists such as those shown in FIG. 10 can be synthesized to maintain the majority of contacts with PYL yet can cause changes in resulting ABI binding surface with a reduced affinity.

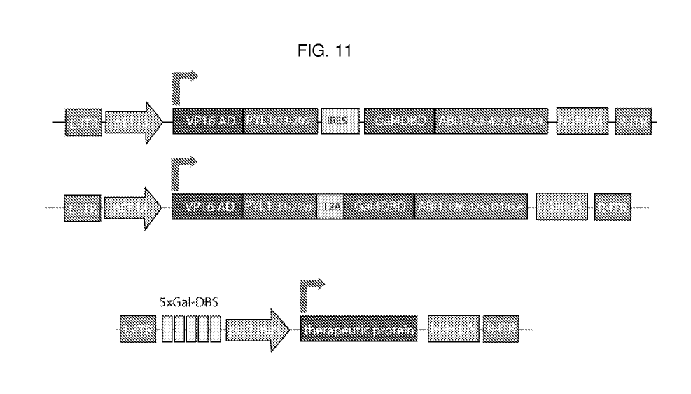

FIG. 11 illustrates adeno-associated virus (AAV) constructs designed for gene activation in mice and in IPS cells.

Based on these studies and the previous evidence that induced proximity can be used to regulate the activity of cellular proteins and receptors by dimerization (e.g., as reported in Spencer D. M., Wandless T. J., Schreiber S. L., and Crabtree G. R., Science 1993, 262, 1019-1024), exchange factors (Holsinger L. J., Spencer D. M., Austin D. J., Schreiber S. L., and Crabtree G. R., Proc Natl Acad Sci USA 1995, 92, 9810-9814), non-receptor tryosine kinase (Graef I. A., Holsinger L. J., Diver S., Schreiber S. L., and Crabtree G. R. EMBO J. 1997, 16, 5618-5628), protein stability (Stankunas K., Bayle J. H., Gestwicki J. E., Lin Y. M., Wandless T. J., and Crabtree G. R., Mol Cell 2003, 12, 1615-1624; Pratt M. R., Schwartz E. C., and Muir T. W., Proc Natl Acad Sci USA 2007, 104, 11209-11214), protein splicing (Mootz H. D. and Muir T. W., J. Am. Chem. Soc. 2002, 124, 9044-9045), protein localization to the cell membrane (Spencer D. M., Graef I., Austin D. J., Schreiber S. L., and Crabtree G. R., Proc Natl Acad Sci USA 1995, 93, 9805-9809; Suh B. C., Inoue T., Meyer T., and Hille B., Science 2006, 314, 1454-1457), protein localization to the nucleus (Belshaw P. J., Ho S. N., Crabtree G. R., and Schreiber S. L., Proc Natl Acad Sci USA 1996, 93, 4604-4607), protein localization to the cytoplasm (Klemm J. D., Beals C. R., and Crabtree G. R., Curr. Biol. 1997, 7, 638-644), protein localization to specific cellular organelle (Komatsu T., Kukelyansky I., McCaffery J. M.; Ueno T., Varela, L. C., and Inoue, T., Nat. Methods 2010, 7, 206-208), protein glycosylation (Kohler J. J. and Bertozzi C. R., Chem. Biol. 2003, 10, 1303-1311), chromosomal segregation (Gruber S., Arumugam P., Katou Y., Kuglitsch D., Helmhart W., Shirahige K., and Nasmyth K., Cell 2006 127, 523-537), and apoptosis (Belshaw P. J., Spencer D. M., Crabtree G. R., and Schreiber S. L., Chem. Biol. 1996 3, 731-738), it is evident that induced dimerization of proximity with ABA is useful for controlling each of these fundamental cellular processes for instructive, investigational and therapeutic purposes.

### III. In-Depth Analysis of the Inducibility of Gene Activation with the ABA-Induced Proximity System

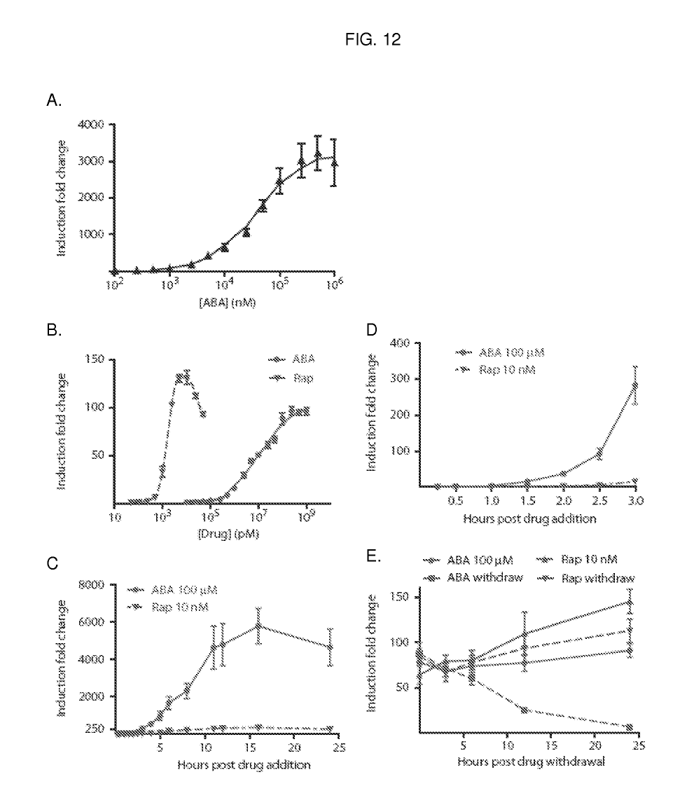

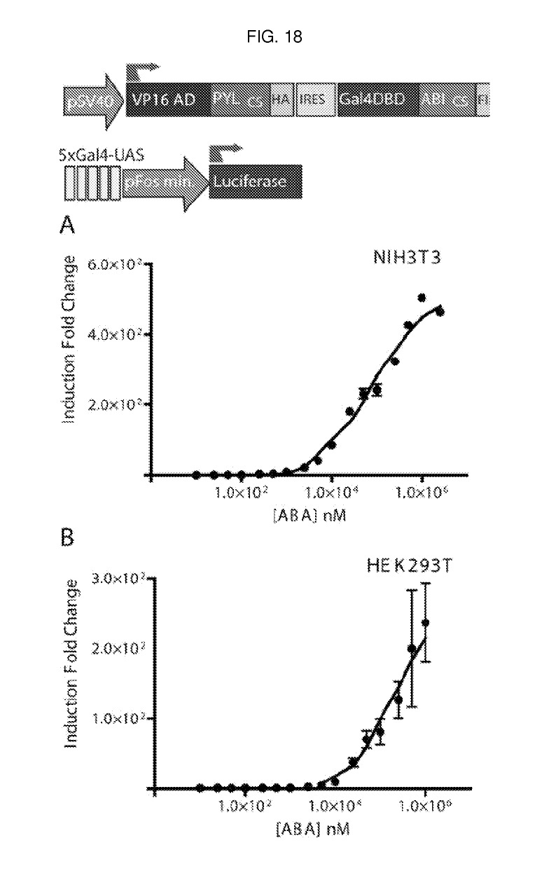

To assay the ability of ABA to induce effective proximity of chimeric proteins, the ability of ABA to reconstitute protein domains was tested. In this strategy, ABA was used to induce the proximity of the yeast Gal4 DNA binding domain (Gal4 DBD) to the herpes simplex virus VP16 transactivation domain (VP16AD) (FIG. 2B). The Gal4 DBD binds specifically to the upstream activation sequence (UAS), but does not activate transcription without a transactivation domain (Giniger, E. and Ptashne, M. (1987) Transcription in yeast activated by a putative amphipathic α helix linked to a DNA binding unit. Nature 330, 670-672). The VP16AD, when tethered to DNA binding domains, strongly activates transcription but cannot act without proximity to a DNA binding domain. To test gene activation by ABA-induced proximity of Gal4 DBD and VP16AD, an ABA-activator cassette was constructed by fusing Gal4 DBD to the domain of ABI1 that interacts with PYL1 (the complementary surface of ABI1, or “ABIcs”) and VP16AD to the domain of PYL1 that interacts with ABI1 (the complementary surface of PYL1, or “PYLcs”) (FIG. 2B). When the ABA-activator cassette was cotransfected with a UAS-luciferase reporter into murine embryonic stem (ES) cells (FIG. 12A) or NIH 3T3 and human embryonic kidney (HEK) 293T cells (FIG. 18), ABA induced luciferase production by more than three orders of magnitude (FIG. 12A).

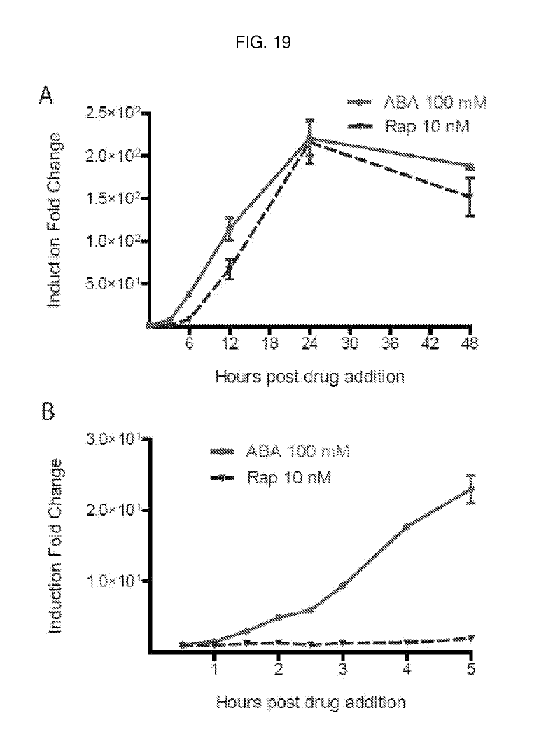

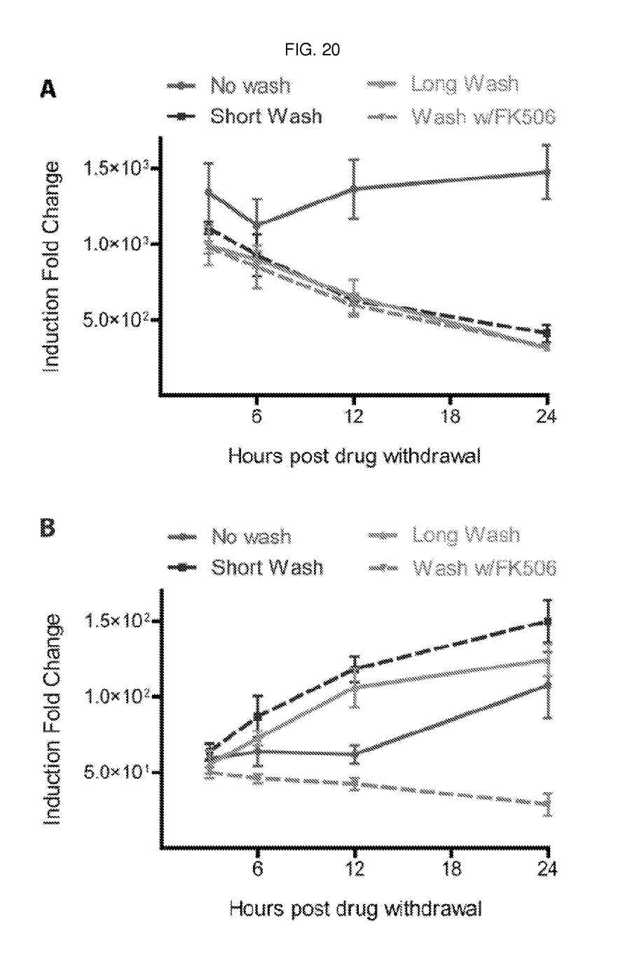

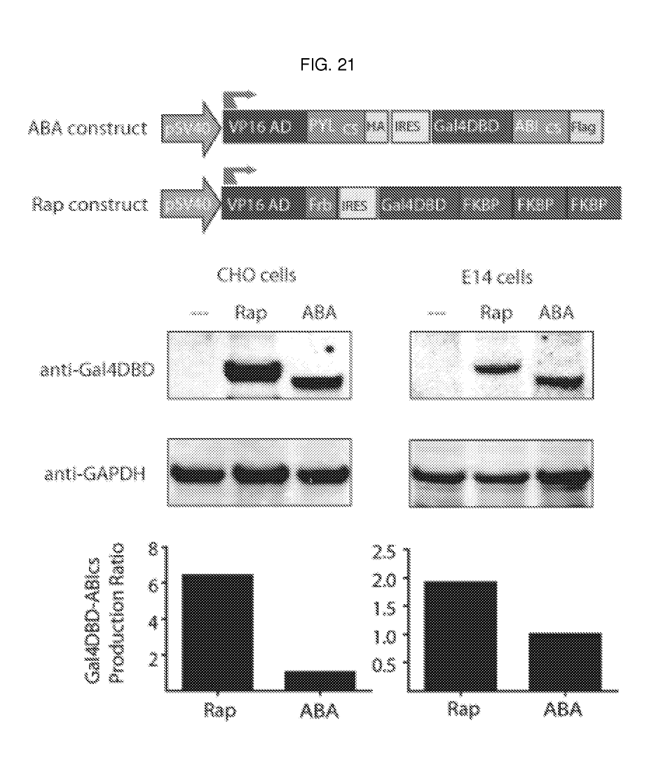

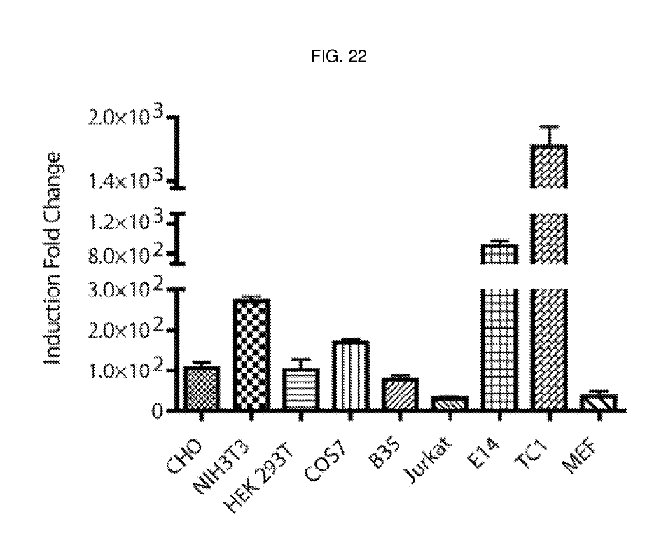

The ABA-induced domain reconstitution leading to induction of gene expression was compared to a similar system in Chinese hamster ovary (CHO) cells controlled by Rap, which has wild-type FKBP12 and Frb replacing ABIcs and PYLcs, respectively, in the chimeric proteins. In contrast to the large linear range of ABA responsiveness (FIG. 12B), Rap-induced transactivation exhibited an essentially “off-on switch” over a factor of 10 concentration change (FIG. 12B). Such a linear dose response of ABA-induced transactivation has the potential to provide precise control of therapeutic protein abundance induced by ABA. The relative activation change induced by ABA was much higher than that by Rap in E14 cells (FIG. 12C) but was comparable in CHO cells (FIG. 12B and FIG. 19). Activation of luciferase was first observed after 1.5 hours of ABA addition and reached a maximum within 24 hours, which was faster than that observed in the Rap system (FIG. 12D and FIG. 19) and is most likely due to the lack of competition with endogenous FKBP. To evaluate the reversibility of the ABA system, cells were first induced to produce luciferase for 24 hours, after which the ABA was withdrawn by washing cells five times with fresh medium without ABA and incubating cells at 37° C. for 5 min between each wash. ABA-induced gene activation was reversed to background within 24 hours in CHO cells (FIG. 12E). On the contrary, the same washing conditions did not reverse Rap-dependent activation over the same time period. A more extensive washing or the addition of competitive binders of Rap against FKBP, such as FK506, is required to reverse Rap-dependent activation (FIG. 20). The observed differences in the inducible gene activation between ABA and Rap may not simply be due to differences in fusion protein production (FIG. 21) but may also result from the combination of differences in mechanisms and affinities of inducer-fusion protein recognition, as well as their association-dissociation rates. ABA-induced gene activation was observed in all cell types tested (FIG. 22).

Controlling Protein Localization and Signal Transduction with the ABA-Induced Proximity System.

The activities of proteins are often regulated by their subcellular localization. In another variation of domain reconstitution, the ability of ABA-induced proximity to control the localization of a chosen protein was tested. Fusions of ABIcs to specific subcellular localization domains were constructed and ABA was used to dimerize these ABIcs localization fusion proteins to green fluorescent protein (GFP)-fused PYLcs. The relocation of GFP-PYLcs to four subcellular locations was induced by fusing ABIcs to (i) Numb to serve as a cytoplasmic localization partner (Dho, S. E. et al. (1999) Characterization of four mammalian Numb protein isoforms: Identification of cytoplasmic and membrane-associated variants of the phosphotyrosine binding domain. J. Biol. Chem. 274, 33097-33104), (ii) to a nuclear localization sequence (NLS) or to Brg1 (Brm/SWI2-related gene 1) to serve as nuclear localization partners (Khavari, P. A. et al. (1993) BRG1 contains a conserved domain of the SWI2/SNF2 family necessary for normal mitotic growth and transcription. Nature 366, 170-174), (iii) to CD4 (cluster of differentiation 4) to serve as a plasma membrane localization partner (Sattentau, Q. J. et al. (1988) The CD4 antigen: Physiological ligand and HIV receptor. Cell 52, 631-633), or (iv) to a myristoylation (myr) sequence to serve as a plasma membrane and an intracellular membrane localization partner (Holsinger, L. J. et al. (1995) Signal transduction in T lymphocytes using a conditional allele of Sos. Proc. Natl. Acad. Sci. U.S.A. 92, 9810-9814)).

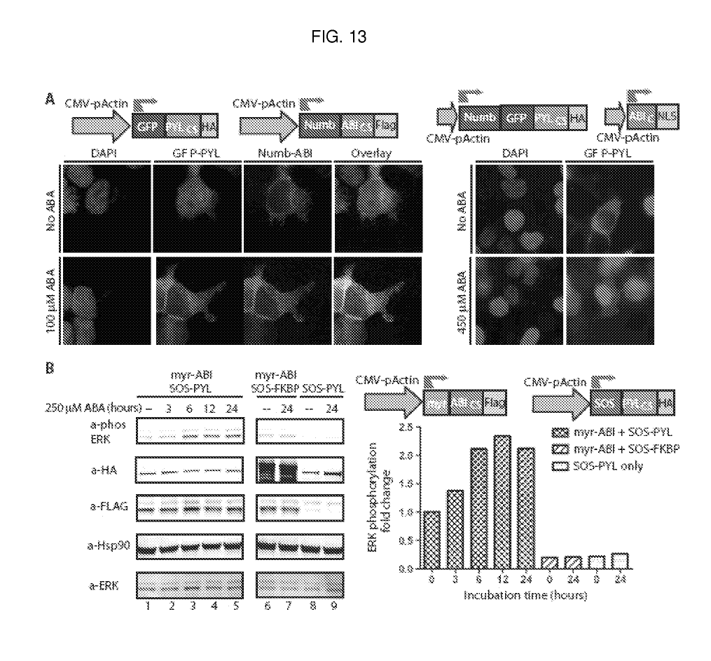

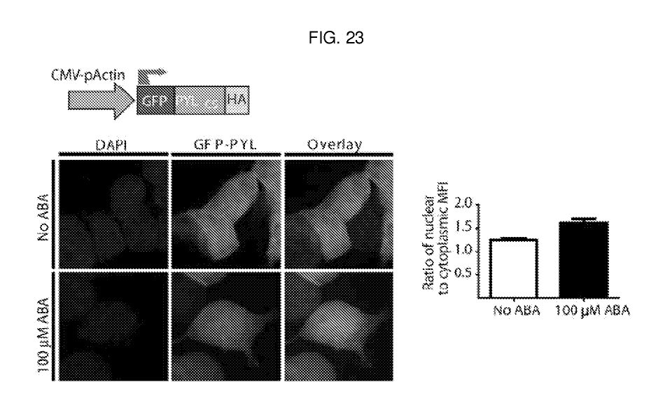

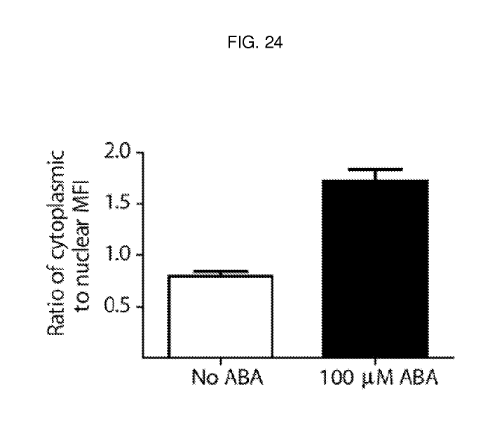

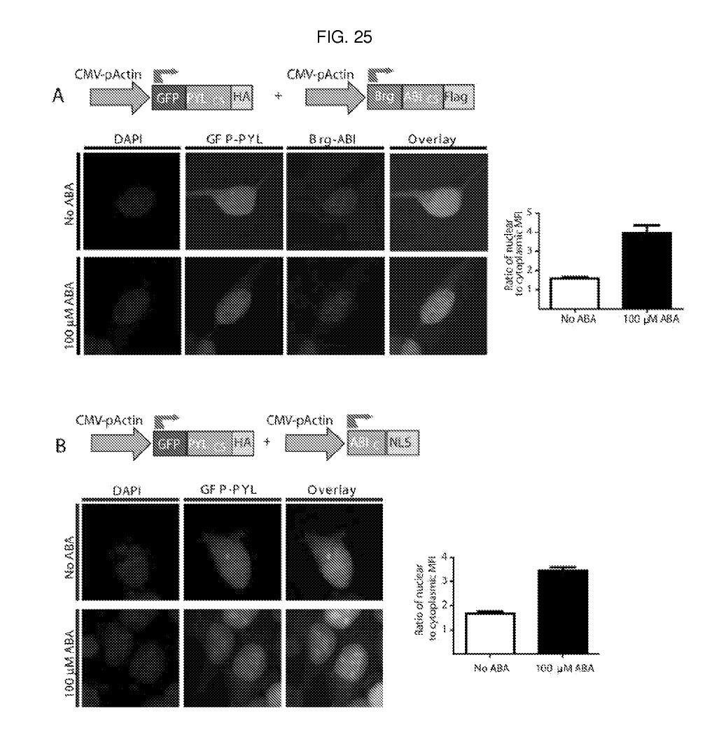

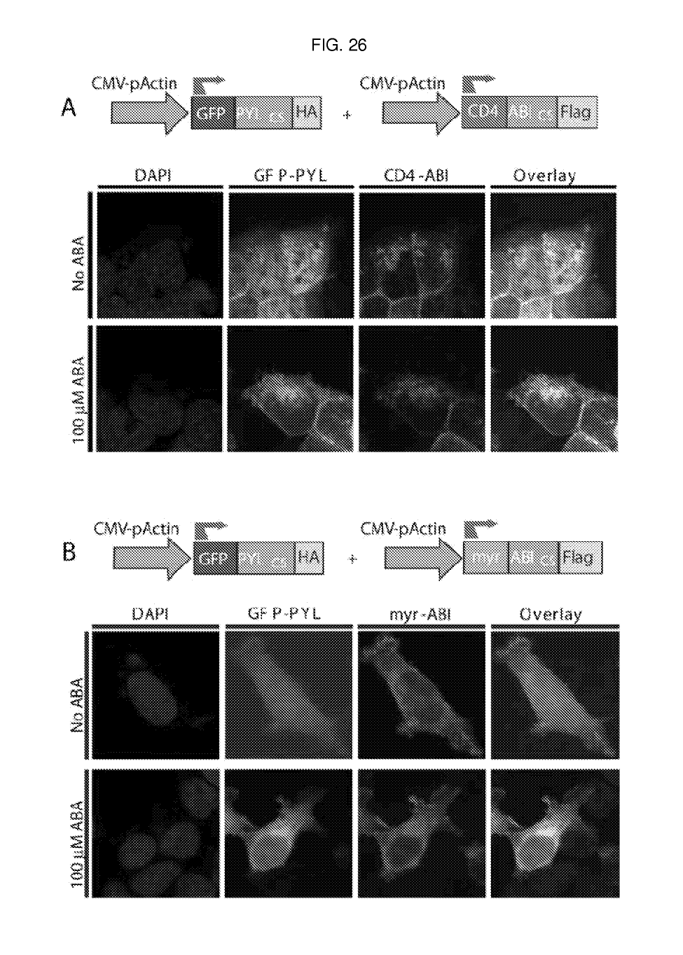

GFP-PYLcs alone showed a pan-cellular localization with or without ABA (FIG. 23), whereas when cytoplasmic Numb-ABIcs was also present, within 30 min ABA induced the cytoplasmic accumulation of GFP-PYLcs at the expense of its nuclear localization in HEK 293T cells (FIG. 13A, left panel, and FIG. 24). When Numb was replaced with either the nuclear protein Brg1 or the membrane protein CD4, GFP-PYLcs relocated to the nucleus or to the membrane, respectively, upon ABA addition within 30 min (FIGS. 25 and 26). The nuclear or membrane localization was also achieved with the NLS-fused ABIcs or the myr-fused ABIcs (FIGS. 25 and 26).

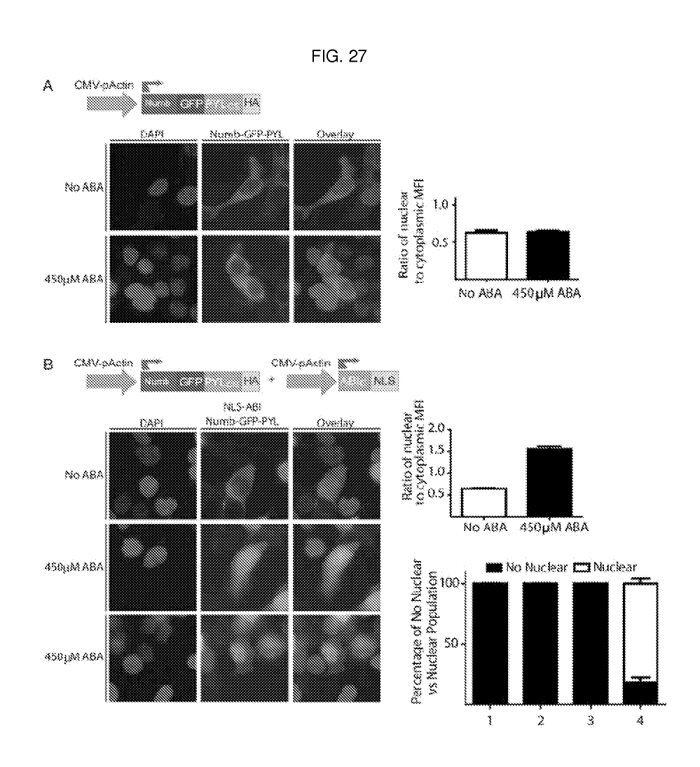

ABA-induced relocalization competed with an endogenous localization mechanism. In the presence of NLS-ABIcs, ABA triggered the redistribution of cytoplasmic Numb-GFP-PYLcs fusion protein into the nucleus (FIG. 13A, right panel, and FIG. 27). The degree of Numb-GFP-PYLcs nuclear localization varied among cells, with many cells showing near-complete localization, as shown in FIG. 27.

In response to growth factor signaling, for example, the guanine nucleotide exchange factor SOS (son of sevenless) moves to the membrane where it activates the guanosine triphosphatase Ras, resulting in activation of the mitogen-activated protein kinase (MAPK) pathway and phosphorylation of extracellular signal-regulated kinases 1 and 2 (ERK1/2) (Mor, A. and Philips, M. R. (2006) Compartmentalized Ras/MAPK signaling. Annu. Rev. Immunol. 24, 771-800). Localization of SOS depends on the binding of its SH2 domains to phosphotyrosines in the adaptor protein GRB2 (growth factor receptor-bound protein 2) or other membrane-localized proteins. ABA induced the phosphorylation of ERK in cells expressing SOS-PYLcs chimeric proteins and myr-ABIcs (FIG. 13B), reflecting relocation of SOS-PYLcs to the plasma membrane. The induction of ERK phosphorylation was not observed when SOS-FKBP fusion protein was used instead of SOS-PYLcs. These studies indicate that ABA can be used to rapidly relocalize proteins and thereby activate or inhibit their functions.

Combining ABA- and Rap-Induced Proximity Systems to Control Independent Biological Processes.

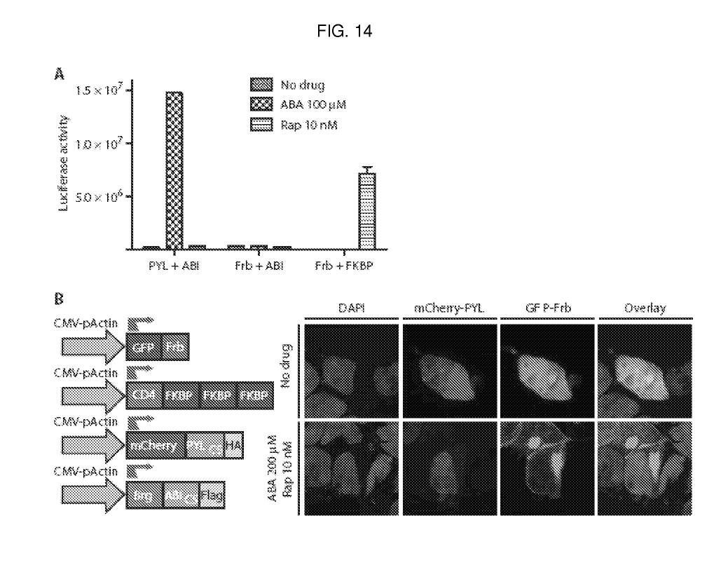

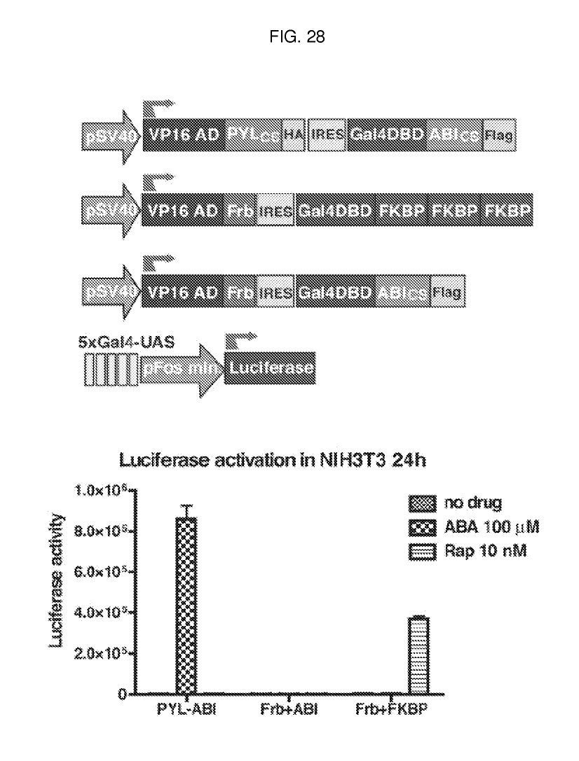

To assess the specificity and independence of ABA- and Rap-induced proximity and thereby their potential to construct genetic circuits with Boolean logic, cells expressing VP16-PYLcs and Gal4 DBD-ABIcs, VP16-Frb and Gal4 DBD-ABIcs, or VP16-Frb and Gal4 DBD-FKBP transgenes, along with the UAS-luciferase reporter, were treated with either ABA or Rap. Luciferase production was only induced by matching small-molecule and dimerizing protein pairs (FIG. 14A and FIG. 28). This result illustrates the potential of using both ABA and Rap as chemical inducers to regulate the expression of two individual genes in the same cell or organism.

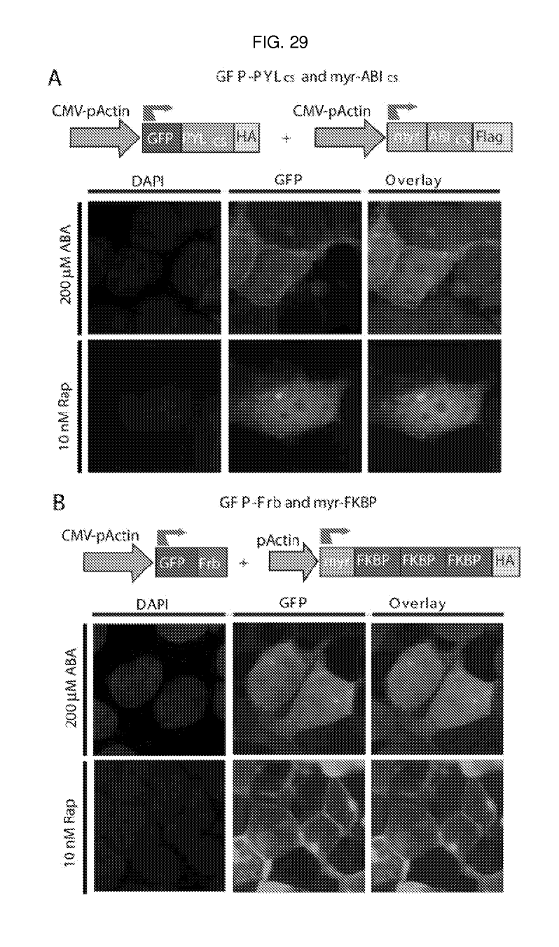

To explore whether ABA and Rap can be used independently to relocate different cellular proteins, 293T cells were transfected with either GFP-PYLcs and myr-ABIcs or GFP-Frb and myr-FKBP. The membrane localization of GFP proteins was observed only when the matching small molecule was added (FIG. 29).

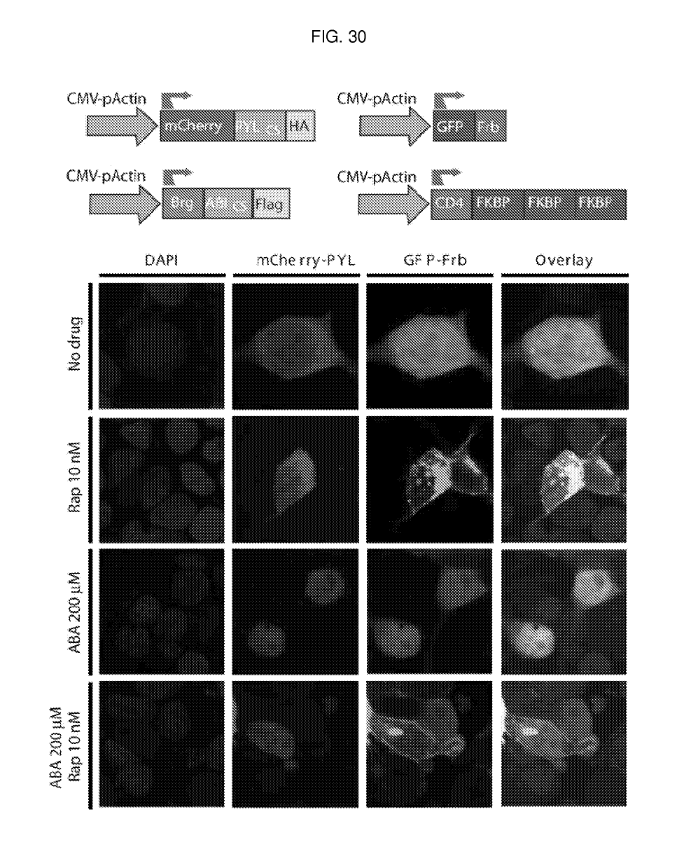

One of the advantages of having orthogonal CIP systems is the ability to manipulate two proteins individually at the same time in the same cell. To demonstrate this application, mCherry-PYLcs and Brg-ABIcs were introduced together with GFP-Frb and CD4-FKBP into 293T cells. When transfected cells were treated with ABA or Rap alone, mCherry-PYLcs or GFP-Frb proteins were recruited to the nucleus or membrane, respectively (FIG. 30). When both small molecules were added, each of these two pan-cellular distributed proteins was relocalized to the desired subcellular compartment within 30 min (FIG. 14B). Thus, the ABA system can be used together with the Rap system to regulate two cellular processes at the same time.

Engineering a Phosphatase-Free ABA-C/P System.

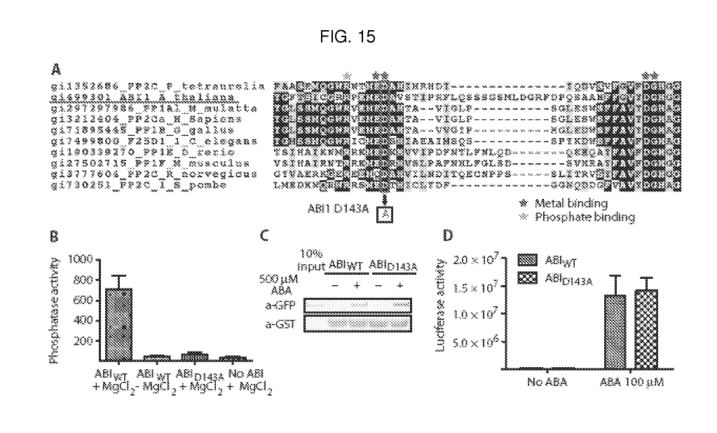

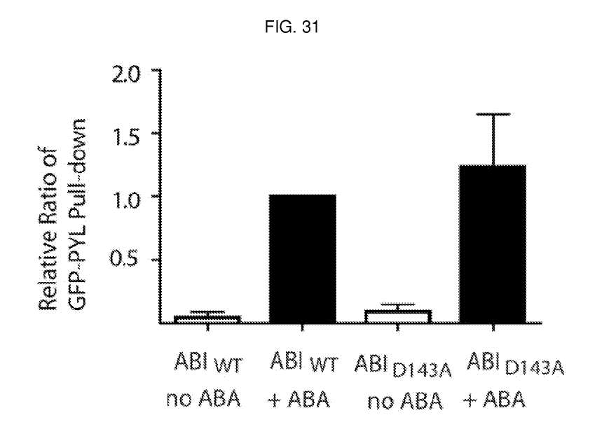

ABA is present in many foods containing plant extracts and oils. Its lack of toxicity is supported by an extensive evaluation by the Environmental Protection Agency (EPA) (Environmental Protection Agency, S-Abscisic acid, (S)-5-(1-hydroxy-2,6,6-trimethyl-4-oxo-1-cyclohex-2-enyl)-3-methyl-penta-(2Z,4E)-dienoic acid; amendment to an exemption from the requirement of a tolerance. Fed. Regist. 75, 11740-11744 (2010)). Although no toxic effects of the ABA-CIP system were observed in cultured mammalian cells, a potential problem is the phosphatase domain of ABI1, which is present in the constructs used herein. To avoid any potential long-term toxic effects when used in animal models or in humans, an ABIcs domain was engineered that lacks the phosphatase activity that maintains the ABA-induced PYLcs binding ability. We made and purified the recombinant glutathione S-transferase (GST) fusion proteins of ABIcs in which Asp143 is mutated to Ala (FIG. 15A). The mutant protein showed phosphatase activity close to background (FIG. 6A and FIG. 15B). Using wild-type or mutant GST-ABIcs to pull down GFP-PYLcs from whole-cell lysates of GFP-PYLcs transgene-expressing 293T cells, the D143A mutant showed similar ABA-dependent PYL binding relative to the wild-type ABI (FIG. 15C and FIG. 31). When the same point mutation was introduced into the ABA-activator cassette used previously and tested its ability to induce luciferase production upon ABA addition, the mutant showed induction comparable to that of the wild-type ABIcs (FIG. 6B and FIG. 15D). These results indicate that the phosphatase activity of ABIcs can be eliminated without sacrificing induction efficiency; therefore, unregulated phosphatase activity should not be an issue with the experimental or therapeutic use of this system.

Assessing ABA Stability, Oral Availability, and Toxicity.

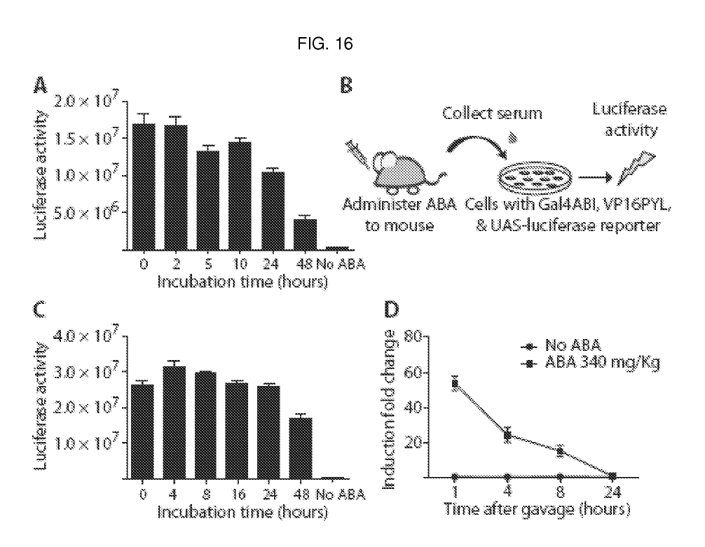

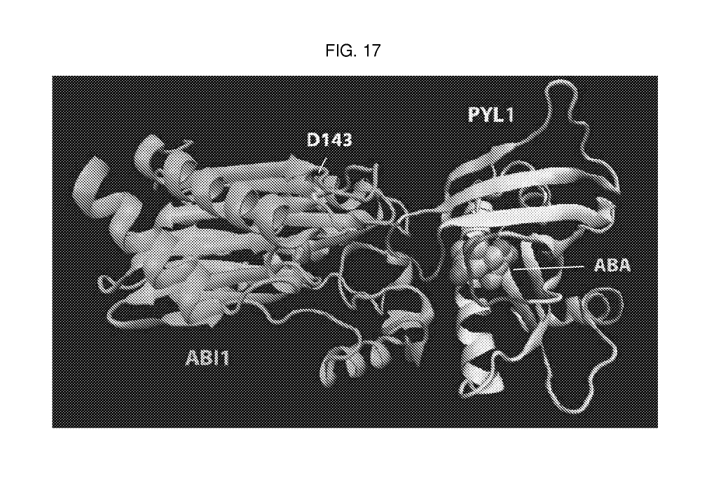

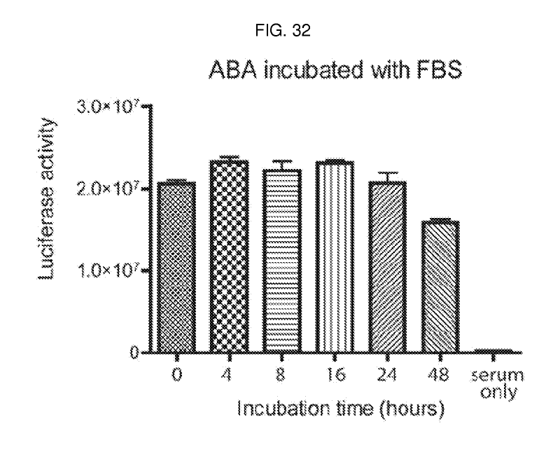

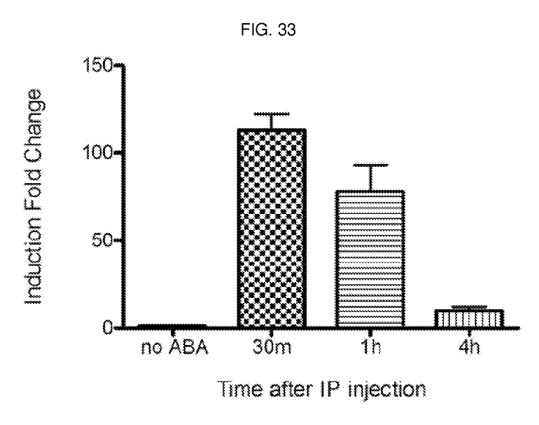

In plants, ABA had been reported to be unstable due to active enzymatic degradation (Cutler, A. J. and Krochko, J. E. (1999) Formation and breakdown of ABA. Trends Plant Sci. 4, 472-478). To examine its stability in mammalian cells, ABA was incubated with CHO cells for up to 48 hours, after which the functional ABA concentration was assayed by the ability of the ABA-incubated cell culture medium to induce luciferase production in cells expressing VP16-PYLcs and wild-type Gal4 DBD-ABIcs. After 24 hours of incubation with cells, about 60% of ABA activity was retained, and after 48 hours, the activity was reduced to around 23% (FIG. 16A), indicating that ABA has a desirable half-life in mammalian cells. For potential therapeutic application in humans, the stability of ABA in the serum and its bioavailability in mice was examined (FIG. 16B). The activity of residual ABA in the serum was measured by its ability to induce the transcriptional activation of the luciferase reporter in cells expressing the ABA-activator cassette. The stability of ABA in isolated serum was tested by incubating ABA with fresh human serum or heat-inactivated fetal bovine serum (FBS) for up to 48 hours and then applying the serum as the ABA source to activate luciferase. ABA was found to be stable in serum for up to 48 hours, retaining about 64 to 77% activity (FIG. 16C and FIG. 32). Using this assay, the bioavailability in mice was evaluated. When ABA was injected intraperitoneally, it entered circulation rapidly, and activity was detectable within 30 min (FIG. 33). When ABA was administered orally, it had a half-life of about 4 hours (FIG. 16D).

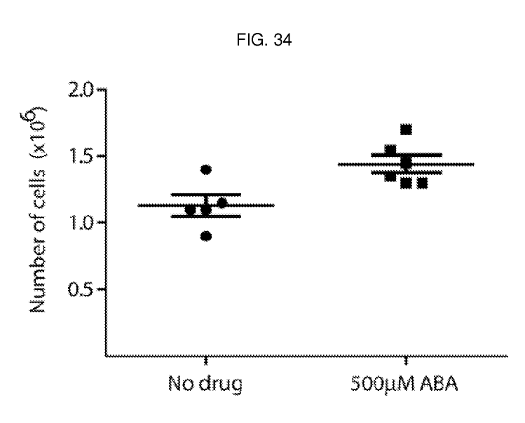

Although ABA has been shown to be nontoxic by the EPA (Environmental Protection Agency, S-Abscisic acid, (S)-5-(1-hydroxy-2,6,6-trimethyl-4-oxo-1-cyclohex-2-enyl)-3-methyl-penta-(2Z,4E)-dienoic acid; amendment to an exemption from the requirement of a tolerance. Fed. Regist. 75, 11740-11744 (2010)), potential toxicity was tested using a lymphocyte proliferation assay. No statistically significant effect upon proliferation induced by antigen receptor signaling was observed (FIG. 34). These studies indicate that ABA is orally bioavailable, its half-life is favorable, and it is likely to be nontoxic in vivo.

The above studies indicate that components of the ABA signaling pathway from Arabidopsis thaliana can be engineered to induce proximity of intracellular proteins and thereby regulate many diverse cellular processes with exogenously applied ABA. The ABA-CIP system that seems well suited for these purposes because, ABA is stable, inexpensive, and nontoxic to cultured cells and mice, and has favorable pharmacokinetics in mice.

ABA is present in our daily consumption of fruits and vegetables; for example, avocados contain 0.76 mg of ABA per kilogram of fresh weight, and no toxic effects to humans have been associated with consumption of ABA to date. ABA has an acute oral median lethal dose (LD50) of >5000 mg/kg in rat and a “no observable adverse effect level” (NOAEL) of 20,000 mg/kg per day in subchronic toxicity studies reported by the EPA. These amounts are much higher than the amounts of ABA used in the above studies. Compared to the original dimerizers, FK1012, FK506, or Rap, ABA is harmless. FK506 is reported to be a powerful inhibitor of T lymphocyte activation (Sawada, S. et al. (1987) Novel immunosuppressive agent FK506: In vitro effects on cloned T cell activation. J Immunol. 139, 1797-1803). Rap has a number of favorable and unfavorable activities, including suppression of interleukin-2-driven lymphocyte proliferation and suppression of transplant rejection (Blazar, B. R. et al. (1994) Rapamycin, a potent inhibitor of T-cell function, prevents graft rejection in murine recipients of allogeneic T-cell-depleted donor marrow. Blood 83, 600-609), and inhibition of proliferation of a number of specific tumors (Luan, F. L. et al. (2002) Rapamycin blocks tumor progression: Unlinking immunosuppression from antitumor efficacy. Transplantation 73, 1565-1572). ABA has been shown to be nontoxic by the EPA, and in agreement with that, ABA did not have an adverse effect in a lymphocyte proliferation assay above. The other components in the above ABA-induced proximity system, i.e. the PYL1 and ABI1 fragments from the plant ABA signaling pathway, are also present in our daily diet; therefore, immune tolerance toward these protein fragments may already be established in humans.

ABA was found to be stable in serum over 48 hours and was orally available in mice. Our studies indicate that the ABA system may be used to achieve a factor of >1000 change in protein activity relative to untreated cells. The ABA system was also found to be independent of the Rap-based CIP system, and thus, may be combined with other inducible systems, such as those based on adenosine 5′-triphosphate (ATP) analogs (41), tetracycline (42), or tamoxifen (43), to engineer genetic circuits with Boolean characteristics.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of inducing proximity of first and second chimeric molecules in a cell, the method comprising:
contacting the cell with an amount of an alkenyl substituted cycloaliphatic (ASC) inducer compound effective to induce proximity of the first and second chimeric molecules, wherein the first and second chimeric molecules each comprise an ASC inducer domain and an effector domain.

2. The method according to claim 1, wherein the ASC inducer compound comprises a cycloaliphatic ring substituted with a hydroxyl and/or oxo group.

3. The method according to claim 2, wherein the ASC inducer compound is described by the formula: 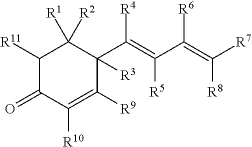
wherein R¹, R², R³, R⁴, R⁵, R⁶, R⁷, R⁸, R⁹, R¹⁰ and R¹¹ are independently selected from hydrogen, an alkyl, an aryl, an alkenyl, an alkynyl, a carbonyl, an acyl, a halogen, a hydroxy, an alkoxy, an aryloxy, and a heterocyclic group and any two of R¹, R², R³, R⁴, R⁵, R⁶, R⁷, R⁸, R⁹, R¹⁰ and R¹¹ can optionally be cyclically linked.

4. The method according to claim 3, wherein the ASC inducer compound is abscisic acid.

5. The method according to claim 1, wherein the first and second chimeric molecules are chimeric proteins.

6. The method according to claim 5, wherein the ASC inducer domain of the first chimeric protein is an ASC inducer compound specific binding domain.

7. The method according to claim 6, wherein the ASC inducer compound specific binding domain of the first chimeric protein comprises a PYR abscisic acid binding domain.

8. The method according to claim 1, wherein the effector domains of the first and second chimeric molecules are different.

9. The method according to claim 1, wherein the effector domain of the first chimeric molecule is a DNA binding domain and the effector domain of the second chimeric molecule is a transcription activation domain.

10. The method according to claim 1, wherein the effector domain of the first chimeric molecule is a cellular localization domain and the effector domain of the second chimeric molecule is a member of a signaling pathway.

11. A nucleic acid comprising a coding sequence for a chimeric protein that comprises ASC inducer domain and an effector domain.

12. The nucleic acid according to claim 11, wherein the ASC inducer domain is an abscisic acid specific binding domain.

13. A kit comprising:
first and second nucleic acids each encoding a chimeric protein that comprises an ASC inducer domain and an effector domain; and
an ASC inducer compound.

14. A composition comprising:
an ASC inducer compound; and
a pharmaceutically acceptable vehicle.

15-16. (canceled)

17. The composition according to claim 14, wherein the ASC inducer compound comprises a cycloaliphatic ring substituted with a hydroxyl and/or oxo group.

18. The composition according to claim 17, wherein the ASC inducer compound is described by the formula: 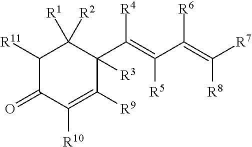
wherein R¹, R², R³, R⁴, R⁵, R⁶, R⁷, R⁸, R⁹, R¹⁰ and R¹¹ are independently selected from hydrogen, an alkyl, an aryl, an alkenyl, an alkynyl, a carbonyl, an acyl, a halogen, a hydroxy, an alkoxy, an aryloxy, and a heterocyclic group and any two of R¹, R², R³, R⁴, R⁵, R⁶, R⁷, R⁸, R⁹, R¹⁰ and R¹¹ can optionally be cyclically linked.

19. The composition according to claim 18, wherein the ASC inducer compound is abscisic acid.
